(19) United States
(12) Patent Application Publication
(10) Pub. No.: US 2022/0016083 A1
(43) Pub. Date: Jan. 20, 2022

(54) METHODS OF TREATING CANCERS

(71) Applicant: Foghorn Therapeutics Inc., Cambridge, MA (US)

(72) Inventors: Richard C. CENTORE, Wakefield, MA (US); Lan XU, Wellesley, MA (US); David LAHR, Watertown, MA (US)

(21) Appl. No.: 17/295,327

(22) PCT Filed: Nov. 21, 2019
(86) PCT No.: PCT/US2019/062525

§ 371 Date: May 19, 2021

Related U.S. Application Data
(60) Provisional application No. 62/858,036, filed on Jun. 6, 2019.
(60) Provisional application No. 62/798,238, filed on Jan. 29, 2019.
(60) Provisional application No. 62/770,446, filed on Nov. 21, 2018.

(51) Int. Cl. A61K 31/4184; A61K 45/06; A61K 31/4439; A61K 31/496; A61K 31/5377; A61P 35/00

(52) U.S. Cl. CPC A61K 31/4184; A61K 45/06; A61P 35/00; A61K 31/496; A61K 31/5377; A61K 31/4439

(57) ABSTRACT

The present invention relates to methods and compositions for the treatment of BAF-related disorders such as melanoma, prostate cancer, breast cancer, bone cancer, renal cell carcinoma, and hematologic cancer.

### BACKGROUND

The invention relates to methods for modulating BRG1- or BRM-associated factors (BAF) complexes for use in the treatment of uveal melanoma or other cancers, e.g., hematologic cancers. In particular, the invention relates to methods for treatment of disorders associated with BAF complex function.

Chromatin regulation is essential for gene expression, and ATP-dependent chromatin remodeling is a mechanism by which such gene expression occurs. The human Switch/Sucrose Non-Fermentable (SWI/SNF) chromatin remodeling complex, also known as BAF complex, has two SWI2-like ATPases known as BRG1 (Brahma-related gene-1) and BRM (Brahma). The transcription activator BRG1, also known as ATP-dependent chromatin remodeler SMARCA4, is encoded by the SMARCA4 gene on chromosome 19. BRG1 is overexpressed in some cancer tumors and is needed for cancer cell proliferation. BRM, also known as probable global transcription activator SNF2L2 and/or ATP-dependent chromatin remodeler SMARCA2, is encoded by the SMARCA2 gene on chromosome 9 and has been shown to be essential for tumor cell growth in cells characterized by loss of BRG1 function mutations. Deactivation of BRG and/or BRM results in downstream effects in cells, including cell cycle arrest and tumor suppression.

Uveal melanoma is a cancer of the eye involving the iris, ciliary body, or chloroid (collectively referred to as the uvea). Tumors arise from the pigment cells (melanocytes) that reside within uvea giving color to the eye. It is the most common primary intraocular malignancy in adults and represents approximately 5 percent of all melanomas recorded in the United States, with approximately 5-6 cases per million people in the United States and Europe. Although 97-98 percent of patients with uveal melanoma have no evidence of metastatic disease at the time of diagnosis and the success rate for local treatment surpasses 90 percent, half of all patients ultimately develop metastatic disease. The 5-year survival rate is about 80% for patients with uveal melanoma confined to the eye and about 15% for patients with metastatic uveal melanoma.

Hematologic cancers, also known as blood cancers, are cancers that begin in blood-forming tissue, such as the bone marrow, or in the cells of the immune system, e.g., leukemias, lymphomas, and myelomas. Leukemias are cancers found in blood and bone marrow which are caused by rapid production of abnormal white blood cells. Lymphomas are cancers which effect the lymphatic system. Myelomas are cancers of the plasma cells. In most hematologic cancers, normal blood cell development is interrupted by uncontrolled growth of an abnormal type of blood cell. The abnormal blood cells prevent the blood from performing many of its functions. Hematologic cancers account for about 10% of all new cancer diagnoses. The 5-year relative survival rates for hematologic cancers range from about 50% to about 90%.

### SUMMARY OF THE INVENTION

The present invention features methods to treat melanoma, prostate cancer, breast cancer, bone cancer, renal cell carcinoma, hematologic cancers, or esophageal cancer, e.g., in a subject in need thereof.

In one aspect, the invention features a method of treating melanoma, prostate cancer, breast cancer, bone cancer, renal cell carcinoma, a hematologic cancer, or esophageal cancer in a subject in need thereof, the method including administering to the subject an effective amount of an agent that reduces the level and/or activity of BRG1 and/or BRM.

In another aspect, the invention features a method of reducing tumor growth of melanoma, prostate cancer, breast cancer, bone cancer, renal cell carcinoma, a hematologic cancer, or esophageal cancer in a subject in need thereof, the method including administering to the subject an effective amount of an agent that reduces the level and/or activity of BRG1 and/or BRM in the tumor.

In another aspect, the invention features a method of suppressing metastatic progression of melanoma, prostate cancer, breast cancer, bone cancer, renal cell carcinoma, a hematologic cancer, or esophageal cancer in a subject, the method including administering an effective amount of an agent that reduces the level and/or activity of BRG1 and/or BRM.

In another aspect, the invention features a method of suppressing metastatic colonization of melanoma, prostate cancer, breast cancer, bone cancer, renal cell carcinoma, a hematologic cancer, or esophageal cancer in a subject, the method including administering an effective amount of an agent that reduces the level and/or activity of BRG1 and/or BRM.

In another aspect, the invention features a method of reducing the level and/or activity of BRG1 and/or BRM in a melanoma, prostate cancer, breast cancer, bone cancer, renal cell carcinoma, hematologic cancer cell, or esophageal cancer cell, the method including contacting the cell with an effective amount of an agent that reduces the level and/or activity of BRG1 and/or BRM in the cell.

In some embodiments of any of the above aspects, the melanoma, prostate cancer, breast cancer, bone cancer, renal cell carcinoma, hematologic cell, or esophageal cell is in a subject.

In some embodiments of any of the above aspects, the effective amount of the agent reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the agent that reduces the level and/or activity of BRG1 by at least 50% (e.g., 55%, 60%, 65%, 70%, 75%, 80%, 85%, 90%, or 95%) as compared to a reference. In some embodiments, the effective amount of the agent that reduces the level and/or activity of BRG1 by at least 90% (e.g., 91%, 92%, 93%, 94%, 95%, 96%, 97%, 98%, or 99%).

In some embodiments, the effective amount of the agent reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the agent that reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments of any of the above aspects, the effective amount of the agent reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the agent that reduces the level and/or activity of BRM by at least 50% (e.g., 55%, 60%, 65%, 70%, 75%, 80%, 85%, 90%, or 95%) as compared to a reference. In some embodiments, the effective amount of the agent that reduces the level and/or activity of BRM by at least 90% (e.g., 91%, 92%, 93%, 94%, 95%, 96%, 97%, 98%, or 99%).

In some embodiments, the effective amount of the agent reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the agent that reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments, the subject has cancer. In some embodiments, the cancer expresses BRG1 and/or BRM protein and/or the cell or subject has been identified as expressing BRG1 and/or BRM. In some embodiments, the cancer expresses BRG1 protein and/or the cell or subject has been identified as expressing BRG1. In some embodiments, the cancer expresses BRM protein and/or the cell or subject has been identified as expressing BRM. In some embodiments, the cancer is melanoma (e.g., uveal melanoma, mucosal melanoma, or cutaneous melanoma). In some embodiments, the melanoma is uveal melanoma. In some embodiments, the cancer is prostate cancer. In some embodiments, the cancer is a hematologic cancer, e.g., multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia (e.g., T-cell acute lymphoblastic leukemia or B-cell acute lymphoblastic leukemia), diffuse large cell lymphoma, or non-Hodgkin's lymphoma. In some embodiments, the cancer is breast cancer (e.g., an ER positive breast cancer, an ER negative breast cancer, triple positive breast cancer, or triple negative breast cancer). In some embodiments, the cancer is a bone cancer (e.g., Ewing's sarcoma). In some embodiments, the cancer is a neuroblastoma. In some embodiments, the cancer is a cutaneous melanoma. In some embodiments, the cancer is a rhabdoid tumor. In some embodiments, the cancer is an upper aerodigestive cancer. In particular embodiments, the cancer is an esophageal cancer (e.g., esophageal adenocarcinoma or esophageal squamous-cell carcinoma). In some embodiments, the cancer is a renal cell carcinoma (e.g., a Microphthalmia Transcription Factor (MITF) family translocation renal cell carcinoma). In some embodiments, the cancer is metastatic (e.g., the cancer has spread to the liver). The metastatic cancer can include cells exhibiting migration and/or invasion of migrating cells and/or include cells exhibiting endothelial recruitment and/or angiogenesis. In other embodiments, the migrating cancer is a cell migration cancer. In still other embodiments, the cell migration cancer is a non-metastatic cell migration cancer. The metastatic cancer can be a cancer spread via seeding the surface of the peritoneal, pleural, pericardial, or subarachnoid spaces. Alternatively, the metastatic cancer can be a cancer spread via the lymphatic system, or a cancer spread hematogenously. In some embodiments, the effective amount of an agent that reduces the level and/or activity of BRG1 and/or BRM is an amount effective to inhibit metastatic colonization of the cancer to the liver.

In some embodiments the cancer harbors a mutation in GNAQ. In some embodiments the cancer harbors a mutation in GNA11. In some embodiments the cancer harbors a mutation in PLCB4. In some embodiments the cancer harbors a mutation in CYSLTR2. In some embodiments the cancer harbors a mutation in BAP1. In some embodiments the cancer harbors a mutation in SF3B1. In some embodiments the cancer harbors a mutation in EIF1AX. In some embodiments the cancer harbors a TFE3 translocation. In some embodiments the cancer harbors a TFEB translocation. In some embodiments the cancer harbors a MTF translocation. In some embodiments the cancer harbors an EZH2 mutation. In some embodiments the cancer harbors a SUZ12 mutation. In some embodiments the cancer harbors an EED mutation.

In some embodiments, the method further includes administering to the subject or contacting the cell with an anticancer therapy, e.g., a chemotherapeutic or cytotoxic agent, immunotherapy, surgery, radiotherapy, thermotherapy, or photocoagulation. In some embodiments, the anticancer therapy is a chemotherapeutic or cytotoxic agent, e.g., an antimetabolite, antimitotic, antitumor antibiotic, asparagine-specific enzyme, bisphosphonates, antineoplastic, alkylating agent, DNA-Repair enzyme inhibitor, histone deacetylase inhibitor, corticosteroid, demethylating agent, immunomodulatory, janus-associated kinase inhibitor, phosphinositide 3-kinase inhibitor, proteasome inhibitor, or tyrosine kinase inhibitor.

In some embodiments, the agent that reduces the level and/or activity of BRG1 and/or BRM is used in combination with another anti-cancer therapy used for the treatment of uveal melanoma such as surgery, a MEK inhibitor, and/or a PKC inhibitor. For example, in some embodiments, the method further comprises performing surgery prior to, subsequent to, or at the same time as administration of the agent that reduces the level and/or activity of BRG1 and/or BRM. In some embodiments, the method further comprises administration of a MEK inhibitor and/or a PKC inhibitor prior to, subsequent to, or at the same time as administration of the agent that reduces the level and/or activity of BRG1 and/or BRM.

In some embodiments, the anticancer therapy and the agent that reduces the level and/or activity of BRG1 and/or BRM in a cell are administered within 28 days of each other and each in an amount that together are effective to treat the subject.

In some embodiments, the subject or cancer has and/or has been identified as having a BRG1 loss of function mutation. In some embodiments, the subject or cancer has and/or has been identified as having a BRM loss of function mutation. In some embodiments, the cancer harbors a BRG1 T91 OM mutation.

In some embodiments, the cancer is resistant to one or more chemotherapeutic or cytotoxic agents (e.g., the cancer has been determined to be resistant to chemotherapeutic or cytotoxic agents such as by genetic markers, or is likely to be resistant, to chemotherapeutic or cytotoxic agents such as a cancer that has failed to respond to a chemotherapeutic or cytotoxic agent). In some embodiments, the cancer has failed to respond to one or more chemotherapeutic or cytotoxic agents. In some embodiments, the cancer is resistant or has failed to respond to dacarbazine, temozolomide, cisplatin, treosulfan, fotemustine, IMCgp100, a CTLA-4 inhibitor (e.g., ipilimumab), a PD-1 inhibitor (e.g., Nivolumab or pembrolizumab), a PD-L1 inhibitor (e.g., atezolizumab, avelumab, or durvalumab), a mitogen-activated protein kinase (MEK) inhibitor (e.g., selumetinib, binimetinib, or tametinib), and/or a protein kinase C (PKC) inhibitor (e.g., sotrastaurin or LXS196, also known as IDE196).

In some embodiments, the cancer is resistant to or failed to respond to a previously administered therapeutic used for the treatment of uveal melanoma such as a MEK inhibitor or PKC inhibitor. For example, in some embodiments, the cancer is resistant to or failed to respond to a mitogen-activated protein kinase (MEK) inhibitor (e.g., selumetinib, binimetinib, or tametinib), and/or a protein kinase C (PKC) inhibitor (e.g., sotrastaurin or LXS196).

In some embodiments, the agent that reduces the level and/or activity of BRG1 and/or BRM in a cell is a small molecule compound, an antibody, an enzyme, and/or a polynucleotide.

In some embodiments, the agent that reduces the level and/or activity of BRG1 and/or BRM in a cell is an enzyme, e.g., a clustered regularly interspaced short palindromic repeats (CRISPR)-associated protein such as CRISPR-associated protein 9 (Cas9), CRISPR-associated protein 12a (Cas12a), a zinc finger nuclease (ZFN), a transcription activator-like effector nuclease (TALEN), or a meganuclease.

In some embodiments, the agent that reduces the level and/or activity of BRG1 and/or BRM in a cell is a polynucleotide, e.g., an antisense nucleic acid, a short interfering RNA (siRNA), a short hairpin RNA (shRNA), a micro RNA (miRNA), a CRISPR/Cas 9 nucleotide, or a ribozyme.

In some embodiments, the agent that reduces the level and/or activity of BRG1 and/or BRM in a cell is a small molecule compound, e.g., a small molecule BRG1 and/or BRM inhibitor. In some embodiments, the agent that reduces the level and/or activity of BRG1 and/or BRM in a cell is a small molecule compound, e.g., a small molecule BRG1 inhibitor. In some embodiments, the agent that reduces the level and/or activity of BRG1 and/or BRM in a cell is a small molecule compound, e.g., a small molecule BRM inhibitor or a degrader.

In some embodiments, the small molecule BRG1 and/or BRM inhibitor is a compound, or pharmaceutically acceptable salt thereof, having the structure of Formula I:

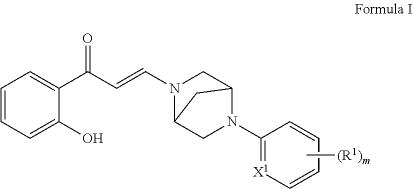

wherein m is 0, 1, 2, 3, or 4;

X¹ is N or CH; and

each R¹ is, independently, independently, halogen, optionally substituted C1-C6 alkyl, optionally substituted C1-C6 heteroalkyl, optionally substituted C3-C10 carbocyclyl, optionally substituted C2-C9 heterocyclyl, optionally substituted C6-C10 aryl, optionally substituted C2-C9 heteroaryl, optionally substituted C2-C6 alkenyl, optionally substituted C2-C6 heteroalkenyl, hydroxy, thiol, or optionally substituted amino.

In some embodiments, the small molecule BRG1 and/or BRM inhibitor is a compound, or pharmaceutically acceptable salt thereof, having the structure of Formula II:

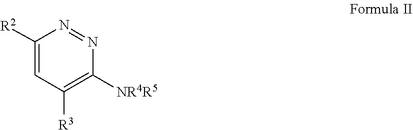

wherein R² is phenyl that is substituted with hydroxy and that is optionally substituted with one or more groups independently selected from the group consisting of halo, cyano, trifluoromethyl, trifluoromethoxy, C₁₋₃ alkyl, and C₁₋₃ alkoxy;

R³ is selected from the group consisting of —R^{a}, —O—R^{a}, —N(R^{a})₂, —S(O)₂R^{a}, and —C(O)—N(R^{a})₂; each R^{a} is, independently, selected from the group consisting of hydrogen, C₁₋₆ alkyl, C₂₋₆ alkenyl, C₂₋₆ alkynyl, 3-15 membered carbocyclyl, and 3-15 membered heterocyclyl, wherein each C₁₋₆ alkyl, C₂₋₆ alkenyl, C₂₋₆ alkynyl, 3-15 membered carbocyclyl, and 3-15 membered heterocyclyl is optionally substituted with one or more groups independently selected from the group consisting of R^{b}, oxo, halo, —NO2, —N(R^{b})₂, —CN, —C(O)—N(R^{b})₂, —S(O)—N(R^{b})₂, —S(O)₂—N(R^{b})₂, —O—R^{b}, —S—R^{b}, —O—C(O)—R^{b}, —C(O)—R^{b}, —C(O)—OR^{b}, —S(O)—R^{b}, —S(O)₂—R^{b}, —N(R^{b})—C(O)—R^{b}, —N(R^{b})—S(O)—R^{b}, —N(R^{b})—C(O)—N(R^{b})₂, and —N(R^{b})—S(O)₂—R^{b};

each R^{b} is, independently, selected from the group consisting of hydrogen, C₁₋₆ alkyl, C₂₋₆ alkenyl, C₂₋₆ alkynyl, C₁₋₆ alkoxy, 3-15 membered carbocyclyl, and 3-15 membered heterocyclyl, wherein each C₁₋₆ alkyl, C₂₋₆ alkenyl, C₂₋₆ alkynyl, C₁₋₆ alkoxy, 3-15 membered carbocyclyl, and 3-15 membered heterocyclyl is optionally substituted with one or more groups independently selected from R^{c}; or two R^{b} are taken together with the nitrogen to which they are attached to form a heterocyclyl that is optionally substituted with one or more groups independently selected from the group consisting of oxo, halo and C₁₋₃ alkyl that is optionally substituted with one or more groups independently selected from the group consisting of oxo and halo;

each R^{c} is, independently, selected from the group consisting of oxo, halo, —NO2, —N(R^{d})₂, —CN, —C(O)—N(R^{d})₂, —S(O)—N(R^{d})₂, —S(O)₂—N(R^{d})₂, —S—R^{d}, —O—C(O)—R^{d}, —C(O)—R^{d}, —C(O)—OR^{d}, —S(O)—R^{d}, —S(O)₂—R^{d}, —N(R^{d})—C(O)—R^{d}, —N(R^{d})—S(O)—R^{d}, —N(R^{d})—C(O)—N(R^{d})₂, —N(R^{d})—S(O)₂— R^{d}, C₁₋₆ alkyl, C₂₋₆ alkenyl, C₂₋₆ alkynyl, 3-15 membered carbocyclyl, and 3-15 membered heterocyclyl, wherein any C₁₋₆ alkyl, C₂₋₆ alkenyl, C₂₋₆ alkynyl, 3-15 membered carbocyclyl, and 3-15 membered heterocyclyl is optionally substituted with one or more groups independently selected from the group consisting of R^{d}, oxo, halo, —NO₂, —N(R^{d})₂, —CN, —C(O)—N(R^{d})₂, —S(O)—N(R^{d})₂, —S(O)₂—N(R^{d})₂, —O—R^{d}, —S—R^{d}, —O—C(O)—R^{d}, —C(O)—R^{d}, —C(O)—R^{d}, —S(O)—R^{d}, —S(O)₂—R^{d}, —N(R^{d})—C(O)—R^{d}, —N(R^{d})—S(O)—R^{d}, —N(R^{d})—C(O)—N(R^{d})₂, and —N(R^{d})—S(O)₂—R^{d};

each R^{d} is, independently, selected from the group consisting of hydrogen, C₁₋₆ alkyl, C₂₋₆ alkenyl, C₂₋₆ alkynyl, carbocyclyl, and carbocyclyl(C₁₋₃ alkyl)-;

R⁴ is H, C₁₋₆ alkyl, or —C(═O)—C₁₋₆ alkyl; and

R⁵ is H or C₁₋₆ alkyl.

Compounds of Formula II may be synthesized by methods known in the art, e.g., those described in U.S. Patent Publication No. 2018/0086720, the synthetic methods of which are incorporated by reference.

In some embodiments, the small molecule BRG1 and/or BRM inhibitor is a compound, or pharmaceutically acceptable salt thereof, having the structure of Formula III:

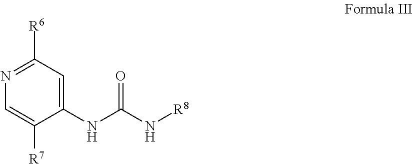

wherein R⁶ is halo, e.g., fluoro or chloro;

R⁷ is hydrogen, optionally substituted amino, or optionally substituted C₁₋₆ alkyl; and

R⁸ is optionally substituted C₆₋₁₀ aryl or optionally substituted C₂₋₉ heteroaryl.

In some embodiments, the small molecule BRG1 and/or BRM inhibitor is a compound, or pharmaceutically acceptable salt thereof, having the structure of any one of compounds 1-16:

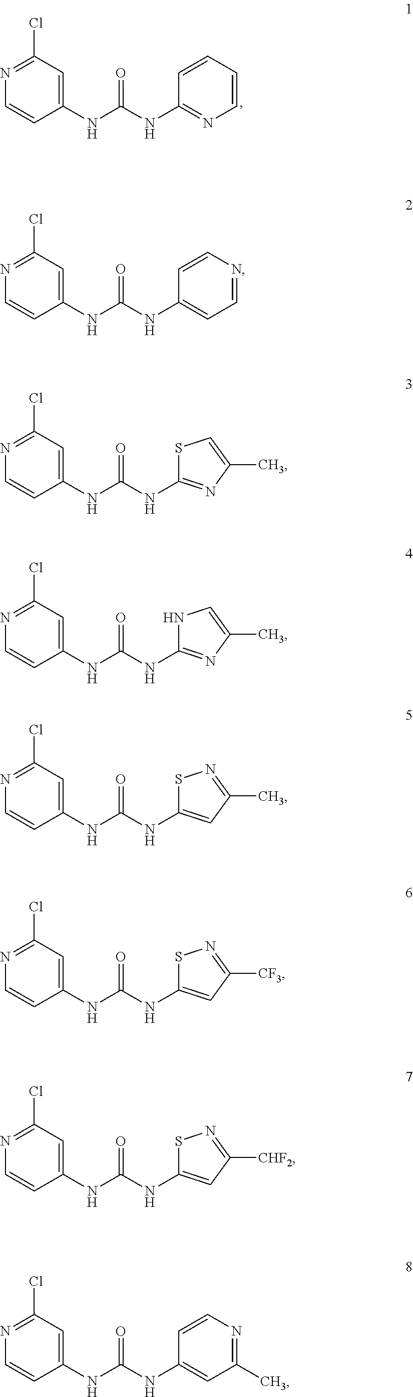

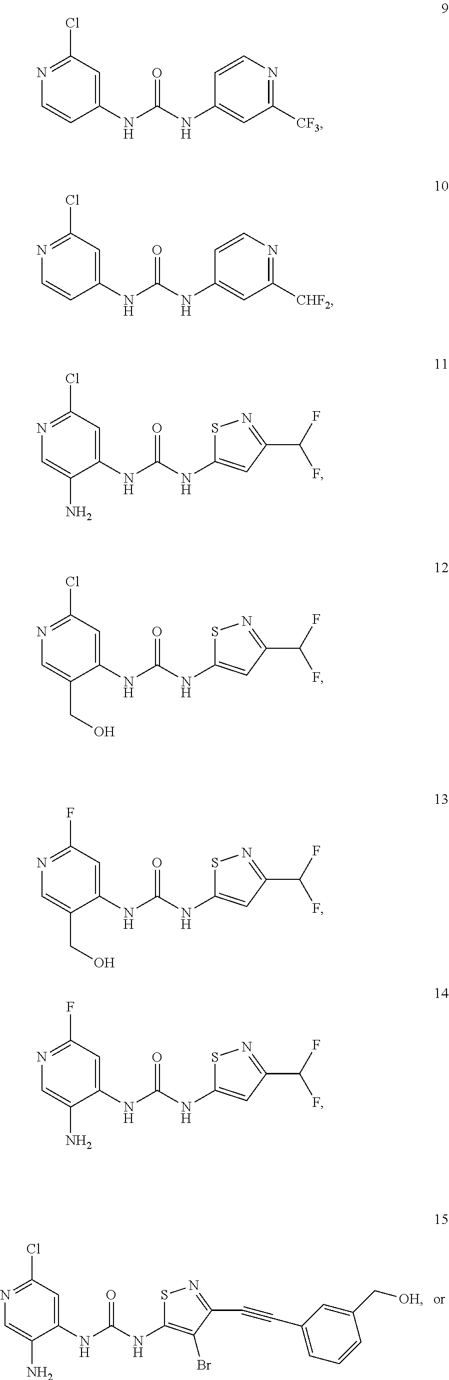

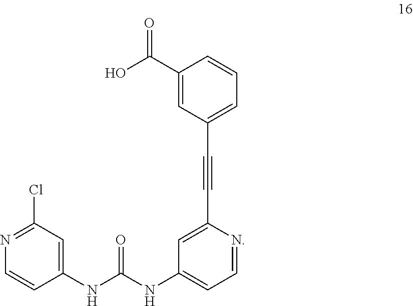 [SMILES: O=C(Nc1ccnc(Cl)c1)Nc1ccnc(C#Cc2cccc(C(=O)O)c2)c1]

In some embodiments, the small molecule compound, or a pharmaceutically acceptable salt thereof is a degrader. In some embodiments, the degrader has the structure of Formula IV:

A-L-B Formula IV

wherein A is a BRG1 and/or BRM binding moiety; L is a linker; and B is a degradation moiety, or a pharmaceutically acceptable salt thereof. In some embodiments, the degradation moiety is a ubiquitin ligase moiety. In some embodiments, the ubiquitin ligase binding moiety includes Cereblon ligands, IAP (Inhibitors of Apoptosis) ligands, mouse double minute 2 homolog (MDM2), hydrophobic tag, or von Hippel-Lindau ligands, or derivatives or analogs thereof.

In some embodiments, A includes the structure of any one of Formula I-III, or any one of compounds 1-16.

In some embodiments, the hydrophobic tag includes a diphenylmethane, adamantine, or tri-Boc arginine, i.e., the hydrophobic tag includes the structure:

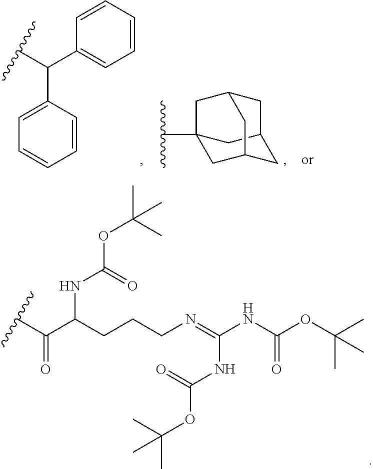 [SMILES: *C(c1ccccc1)c1ccccc1 ; *C12CC3CC(CC(C3)C1)C2 ; *C(=O)C(CCCN=C(NC(=O)OC(C)(C)C)NC(=O)OC(C)(C)C)NC(=O)OC(C)(C)C]

In some embodiments, the ubiquitin ligase binding moiety includes the structure of Formula A:

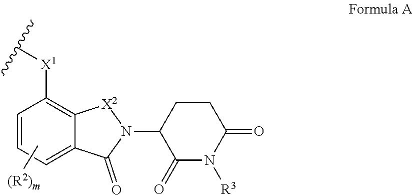

wherein X¹ is CH₂, O, S, or NR¹, wherein R¹ is H, optionally substituted C₁-C₆ alkyl, or optionally substituted C₁-C₆ heteroalkyl; X² is C═O, CH₂, or

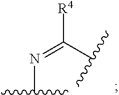

R³ and R⁴ are, independently, H, optionally substituted C₁-C₆ alkyl, or optionally substituted C₁-C₆ heteroalkyl; m is 0, 1, 2, 3, or 4; and each R² is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino,

or a pharmaceutically acceptable salt thereof.

In some embodiments, the ubiquitin ligase binding moiety includes the structure:

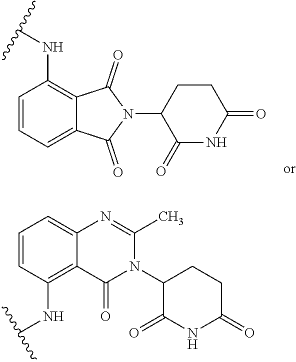

or is a derivative or an analog thereof, or a pharmaceutically acceptable salt thereof.

In some embodiments, the ubiquitin ligase binding moiety includes the structure of Formula B:

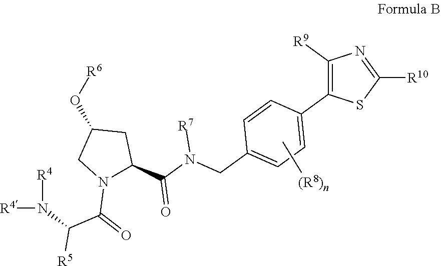

wherein each R⁴, R^{4′}, and R⁷ is, independently, H, optionally substituted C₁-C₆ alkyl, or optionally substituted C₁-C₆ heteroalkyl; R⁵ is optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R⁶ is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; n is 0, 1, 2, 3, or 4; each R⁸ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and each R⁹ and R¹⁰ is, independently, H, halogen, optionally substituted C₁-C₆ alkyl, or optionally substituted C₆-C₁₀ aryl, wherein R^{4′} or R⁵ includes a bond to the linker, or a pharmaceutically acceptable salt thereof.

In some embodiments, the ubiquitin ligase binding moiety includes the structure:

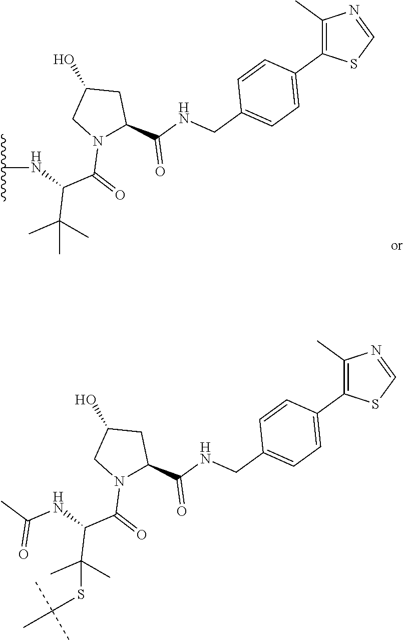

or is a derivative or analog thereof, or a pharmaceutically acceptable salt thereof.

In some embodiments, the ubiquitin ligase binding moiety includes the structure of Formula C:

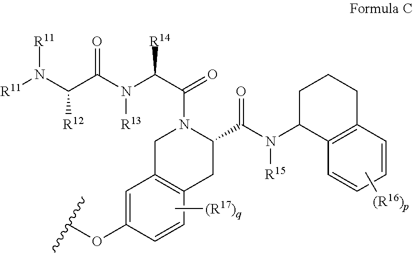

wherein each R¹¹, R¹³, and R¹⁵ is, independently, H, optionally substituted C₁-C₆ alkyl, or optionally substituted C₁-C₆ heteroalkyl; R¹² is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R¹⁴ is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; p is 0, 1, 2, 3, or 4; each R¹⁶ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; q is 0, 1, 2, 3, or 4; and each R¹⁷ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the ubiquitin ligase binding moiety includes the structure:

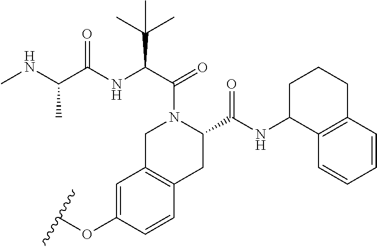 [SMILES: *Oc1ccc2c(c1)CN(C(=O)[C@@H](NC(=O)[C@H](C)NC)C(C)(C)C)[C@H](C(=O)NC1CCCc3ccccc31)C2]

or is a derivative or an analog thereof, or a pharmaceutically acceptable salt thereof.

In some embodiments, the ubiquitin ligase binding moiety includes the structure of Formula D:

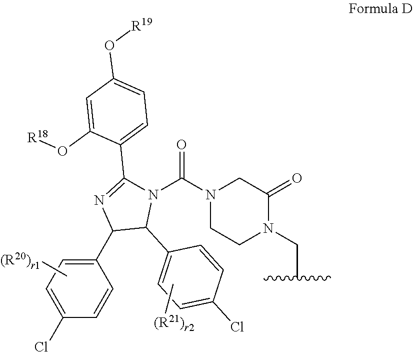

wherein each R¹⁸ and R¹⁹ is, independently, H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; r1 is 0, 1, 2, 3, or 4; each R²⁰ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; r2 is 0, 1, 2, 3, or 4; and each R²¹ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the ubiquitin ligase binding moiety includes the structure:

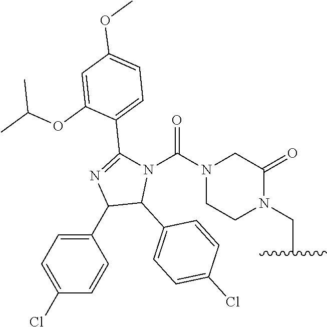 [SMILES: *CN1CCN(C(=O)N2C(c3ccc(OC)cc3OC(C)C)=NC(c3ccc(Cl)cc3)C2c2ccc(Cl)cc2)CC1=O]

or is a derivative or an analog thereof, or a pharmaceutically acceptable salt thereof.

In some embodiments, the linker has the structure of Formula V:

A¹-(B¹)_{f}—(C¹)_{g}—(B²)ₕ-(D)-(B³)ᵢ—(C²)ⱼ—(B⁴)ₖ-A² Formula V

wherein A¹ is a bond between the linker and A; A² is a bond between B and the linker; B¹, B², B³, and B⁴ each, independently, is selected from optionally substituted C₁-C₂ alkyl, optionally substituted C₁-C₃ heteroalkyl, O, S, S(O)₂, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; f, g, h, l, j, and k are each, independently, 0 or 1; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

In some embodiments, D is optionally substituted C₂-C₁₀ polyethylene glycol. In some embodiments, C¹ and C² are each, independently, a carbonyl or sulfonyl. In some embodiments, B¹, B², B³, and B⁴ each, independently, is selected from optionally substituted C₁-C₂ alkyl, optionally substituted C₁-C₃ heteroalkyl, O, S, S(O)₂, and NR^{N}; R^{N} is hydrogen or optionally substituted C₁₋₄ alkyl. In some embodiments, B¹, B², B³, and B⁴ each, independently, is selected from optionally substituted C₁-C₂ alkyl or optionally substituted C₁-C₃ heteroalkyl. In some embodiments, j is 0. In some embodiments, k is 0. In some embodiments, j and k are each, independently, 0. In some embodiments, f, g, h, and i are each, independently, 1.

In some embodiments, the linker of Formula V has the structure of Formula Va:

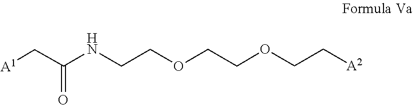

wherein A¹ is a bond between the linker and A, and A² is a bond between B and the linker.

In some embodiments, D is optionally substituted C₁₋₁₀ alkyl. In some embodiments, C₁ and C₂ are each, independently, a carbonyl. In some embodiments, B¹, B², B³, and B⁴ each, independently, is selected from optionally substituted C₁-C₂ alkyl, optionally substituted C₁-C₃ heteroalkyl, O, S, S(O)₂, and NR^{N}, wherein R^{N} is hydrogen or optionally substituted C₁₋₄ alkyl. In some embodiments, B¹, B², B³, and B⁴ each, independently, is selected from optionally substituted C₁-C₂ alkyl, 0, S, S(O)₂, and NR^{N}, wherein R^{N} is hydrogen or optionally substituted C₁₋₄ alkyl. In some embodiments, B¹ and B⁴ each, independently, is optionally substituted C₁-C₂ alkyl. In some embodiments, B¹ and B⁴ each, independently, is C₁ alkyl. In some embodiments, B² and B⁴ each, independently, is NR^{N}, wherein R^{N} is hydrogen or optionally substituted C₁₋₄ alkyl. In some embodiments, B² and B⁴ each, independently, is NH. In some embodiments, f, g, h, l, j, and k are each, independently, 1.

In some embodiments, the linker of Formula V has the structure of Formula Vb:

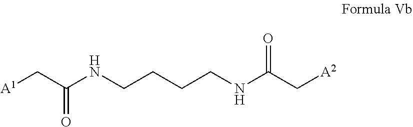

wherein A¹ is a bond between the linker and A, and A² is a bond between B and the linker.

### Chemical Terms

For any of the following chemical definitions, a number following an atomic symbol indicates that total number of atoms of that element that are present in a particular chemical moiety. As will be understood, other atoms, such as hydrogen atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

The term “acyl,” as used herein, represents a hydrogen or an alkyl group that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the compounds described herein can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged polycycloalkyl,” as used herein, refers to a bridged polycyclic group of 5 to 20 carbons, containing from 1 to 3 bridges.

The term “cyano,” as used herein, represents a —CN group.

The term “carbocyclyl,” as used herein, refers to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

The term “halogen,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups.

Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, refers a mono- or polycyclic radical having 3 to 12 atoms having at least one ring containing 1, 2, 3, or 4 ring atoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an —OH group.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds described herein can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on 25 opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds described herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide 35 of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “including” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1-associated or HBRM-associated factors complex in a human cell.

As used herein, the term “BRG1 loss of function mutation” refers to a mutation in BRG1 that leads to the protein having diminished activity (e.g., at least 1% reduction in BRG1 activity, for example 2%, 5%, 10%, 25%, 50%, or 100% reduction in BRG1 activity). Exemplary BRG1 loss of function mutations include, but are not limited to, a homozygous BRG1 mutation and a deletion at the C-terminus of BRG1.

As used herein, the term “BRG1 loss of function disorder” refers to a disorder (e.g., cancer) that exhibits a reduction in BRG1 activity (e.g., at least 1% reduction in BRG1 activity, for example 2%, 5%, 10%, 25%, 50%, or 100% reduction in BRG1 activity).

As used herein, the term “BRM loss of function mutation” refers to a mutation in BRM that leads to the protein having diminished activity (e.g., at least 1% reduction in BRM activity, for example 2%, 5%, 10%, 25%, 50%, or 100% reduction in BRM activity). Exemplary BRM loss of function mutations include, but are not limited to, a homozygous BRM mutation and a deletion at the C-terminus of BRM.

As used herein, the term “BRM loss of function disorder” refers to a disorder (e.g., cancer) that exhibits a reduction in BRM activity (e.g., at least 1% reduction in BRM activity, for example 2%, 5%, 10%, 25%, 50%, or 100% reduction in BRG1 activity).

As used herein, the terms “GBAF complex” and “GBAF” refer to a SWI/SNF ATPase chromatin remodeling complex in a human cell. GBAF complex subunits may include, but are not limited to, ACTB, ACTL6A, ACTL6B, BICRA, BICRAL, BRD9, SMARCA2, SMARCA4, SMARCC1, SMARCD1, SMARCD2, SMARCD3, and SS18.

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the term “BRG1” refers to ATP-dependent chromatin remodeler SMARCA4. BRG1 is a component of the BAF complex, a SWI/SNF ATPase chromatin remodeling complex. Human BRG1 is encoded by the SMARCA4 gene on chromosome 19, a nucleic acid sequence of which is set forth in SEQ ID NO: 1. (GenBank Accession No.: NM_001128849.1 (mRNA); www.ncbi.nlm.nih.gov/nuccore/NM_001128849.1 ?report=fasta)

GGCGGGGGAGGCGCCGGGAAGTCGACGGCGCCGGCGGCTCCTGCAGGAGGCCACTGTCTGCAGCTCCCGT
GAAGATGTCCACTCCAGACCCACCCCTGGGCGGAACTCCTCGGCCAGGTCCTTCCCCGGGCCCTGGCCCT
TCCCCTGGAGCCATGCTGGGCCCTAGCCCGGGTCCCTCGCCGGGCTCCGCCCACAGCATGATGGGGCCCA
GCCCAGGGCCGCCCTCAGCAGGACACCCCATCCCCACCCAGGGGCCTGGAGGGTACCCTCAGGACAACAT
GCACCAGATGCACAAGCCCATGGAGTCCATGCATGAGAAGGGCATGTCGGACGACCCGCGCTACAACCAG
ATGAAAGGAATGGGGATGCGGTCAGGGGGCCATGCTGGGATGGGGCCCCCGCCCAGCCCCATGGACCAGC
ACTCCCAAGGTTACCCCTCGCCCCTGGGTGGCTCTGAGCATGCCTCTAGTCCAGTTCCAGCCAGTGGCCC
GTCTTCGGGGCCCCAGATGTCTTCCGGGCCAGGAGGTGCCCCGCTGGATGGTGCTGACCCCCAGGCCTTG
GGGCAGCAGAACCGGGGCCCAACCCCATTTAACCAGAACCAGCTGCACCAGCTCAGAGCTCAGATCATGG
CCTACAAGATGCTGGCCAGGGGGCAGCCCCTCCCCGACCACCTGCAGATGGCGGTGCAGGGCAAGCGGCC
GATGCCCGGGATGCAGCAGCAGATGCCAACGCTACCTCCACCCTCGGTGTCCGCAACAGGACCCGGCCCT
GGCCCTGGCCCTGGCCCCGGCCCGGGTCCCGGCCCGGCACCTCCAAATTACAGCAGGCCTCATGGTATGG
GAGGGCCCAACATGCCTCCCCCAGGACCCTCGGGCGTGCCCCCCGGGATGCCAGGCCAGCCTCCTGGAGG
GCCTCCCAAGCCCTGGCCTGAAGGACCCATGGCGAATGCTGCTGCCCCCACGAGCACCCCTCAGAAGCTG
ATTCCCCCGCAGCCAACGGGCCGCCCTTCCCCCGCGCCCCCTGCCGTCCCACCCGCCGCCTCGCCCGTGA
TGCCACCGCAGACCCAGTCCCCCGGGCAGCCGGCCCAGCCCGCGCCCATGGTGCCACTGCACCAGAAGCA
GAGCCGCATCACCCCCATCCAGAAGCCGCGGGGCCTCGACCCTGTGGAGATCCTGCAGGAGCGCGAGTAC
AGGCTGCAGGCTCGCATCGCACACCGAATTCAGGAACTTGAAAACCTTCCCGGGTCCCTGGCCGGGGATT
TGCGAACCAAAGCGACCATTGAGCTCAAGGCCCTCAGGCTGCTGAACTTCCAGAGGCAGCTGCGCCAGGA
GGTGGTGGTGTGCATGCGGAGGGACACAGCGCTGGAGACAGCCCTCAATGCTAAGGCCTACAAGCGCAGC
AAGCGCCAGTCCCTGCGCGAGGCCCGCATCACTGAGAAGCTGGAGAAGCAGCAGAAGATCGAGCAGGAGC
GCAAGCGCCGGCAGAAGCACCAGGAATACCTCAATAGCATTCTCCAGCATGCCAAGGATTTCAAGGAATA
TCACAGATCCGTCACAGGCAAAATCCAGAAGCTGACCAAGGCAGTGGCCACGTACCATGCCAACACGGAG
CGGGAGCAGAAGAAAGAGAACGAGCGGATCGAGAAGGAGCGCATGCGGAGGCTCATGGCTGAAGATGAGG
AGGGGTACCGCAAGCTCATCGACCAGAAGAAGGACAAGCGCCTGGCCTACCTCTTGCAGCAGACAGACGA
GTACGTGGCTAACCTCACGGAGCTGGTGCGGCAGCACAAGGCTGCCCAGGTCGCCAAGGAGAAAAAGAAG
AAAAAGAAAAAGAAGAAGGCAGAAAATGCAGAAGGACAGACGCCTGCCATTGGGCCGGATGGCGAGCCTC
TGGACGAGACCAGCCAGATGAGCGACCTCCCGGTGAAGGTGATCCACGTGGAGAGTGGGAAGATCCTCAC
AGGCACAGATGCCCCCAAAGCCGGGCAGCTGGAGGCCTGGCTCGAGATGAACCCGGGGTATGAAGTAGCT
CCGAGGTCTGATAGTGAAGAAAGTGGCTCAGAAGAAGAGGAAGAGGAGGAGGAGGAAGAGCAGCCGCAGG
CAGCACAGCCTCCCACCCTGCCCGTGGAGGAGAAGAAGAAGATTCCAGATCCAGACAGCGATGACGTCTC
TGAGGTGGACGCGCGGCACATCATTGAGAATGCCAAGCAAGATGTCGATGATGAATATGGCGTGTCCCAG
GCCCTTGCACGTGGCCTGCAGTCCTACTATGCCGTGGCCCATGCTGTCACTGAGAGAGTGGACAAGCAGT
CAGCGCTTATGGTCAATGGTGTCCTCAAACAGTACCAGATCAAAGGTTTGGAGTGGCTGGTGTCCCTGTA
CAACAACAACCTGAACGGCATCCTGGCCGACGAGATGGGCCTGGGGAAGACCATCCAGACCATCGCGCTC
ATCACGTACCTCATGGAGCACAAACGCATCAATGGGCCCTTCCTCATCATCGTGCCTCTCTCAACGCTGT
CCAACTGGGCGTACGAGTTTGACAAGTGGGCCCCCTCCGTGGTGAAGGTGTCTTACAAGGGATCCCCAGC
AGCAAGACGGGCCTTTGTCCCCCAGCTCCGGAGTGGGAAGTTCAACGTCTTGCTGACGACGTACGAGTAC
ATCATCAAAGACAAGCACATCCTCGCCAAGATCCGTTGGAAGTACATGATTGTGGACGAAGGTCACCGCA
TGAAGAACCACCACTGCAAGCTGACGCAGGTGCTCAACACGCACTATGTGGCACCCCGCCGCCTGCTGCT
GACGGGCACACCGCTGCAGAACAAGCTTCCCGAGCTCTGGGCGCTGCTCAACTTCCTGCTGCCCACCATC
TTCAAGAGCTGCAGCACCTTCGAGCAGTGGTTTAACGCACCCTTTGCCATGACCGGGGAAAAGGTGGACC
TGAATGAGGAGGAAACCATTCTCATCATCCGGCGTCTCCACAAAGTGCTGCGGCCCTTCTTGCTCCGACG
ACTCAAGAAGGAAGTCGAGGCCCAGTTGCCCGAAAAGGTGGAGTACGTCATCAAGTGCGACATGTCTGCG
CTGCAGCGAGTGCTCTACCGCCACATGCAGGCCAAGGGCGTGCTGCTGACTGATGGCTCCGAGAAGGACA
AGAAGGGCAAAGGCGGCACCAAGACCCTGATGAACACCATCATGCAGCTGCGGAAGATCTGCAACCACCC
CTACATGTTCCAGCACATCGAGGAGTCCTTTTCCGAGCACTTGGGGTTCACTGGCGGCATTGTCCAAGGG
CTGGACCTGTACCGAGCCTCGGGTAAATTTGAGCTTCTTGATAGAATTCTTCCCAAACTCCGAGCAACCA
ACCACAAAGTGCTGCTGTTCTGCCAAATGACCTCCCTCATGACCATCATGGAAGATTACTTTGCGTATCG
CGGCTTTAAATACCTCAGGCTTGATGGAACCACGAAGGCGGAGGACCGGGGCATGCTGCTGAAAACCTTC
AACGAGCCCGGCTCTGAGTACTTCATCTTCCTGCTCAGCACCCGGGCTGGGGGGCTCGGCCTGAACCTCC
AGTCGGCAGACACTGTGATCATTTTTGACAGCGACTGGAATCCTCACCAGGACCTGCAAGCGCAGGACCG
AGCCCACCGCATCGGGCAGCAGAACGAGGTGCGTGTGCTCCGCCTCTGCACCGTCAACAGCGTGGAGGAG
AAGATCCTAGCTGCAGCCAAGTACAAGCTCAACGTGGACCAGAAGGTGATCCAGGCCGGCATGTTCGACC
AGAAGTCCTCCAGCCATGAGCGGCGCGCCTTCCTGCAGGCCATCCTGGAGCACGAGGAGCAGGATGAGAG
CAGACACTGCAGCACGGGCAGCGGCAGTGCCAGCTTCGCCCACACTGCCCCTCCGCCAGCGGGCGTCAAC
CCCGACTTGGAGGAGCCACCTCTAAAGGAGGAAGACGAGGTGCCCGACGACGAGACCGTCAACCAGATGA
TCGCCCGGCACGAGGAGGAGTTTGATCTGTTCATGCGCATGGACCTGGACCGCAGGCGCGAGGAGGCCCG
CAACCCCAAGCGGAAGCCGCGCCTCATGGAGGAGGACGAGCTCCCCTCGTGGATCATCAAGGACGACGCG
GAGGTGGAGCGGCTGACCTGTGAGGAGGAGGAGGAGAAGATGTTCGGCCGTGGCTCCCGCCACCGCAAGG
AGGTGGACTACAGCGACTCACTGACGGAGAAGCAGTGGCTCAAGAAAATTACAGGAAAAGATATCCATGA
CACAGCCAGCAGTGTGGCACGTGGGCTACAATTCCAGCGTGGCCTTCAGTTCTGCACACGTGCGTCAAAG
GCCATCGAGGAGGGCACGCTGGAGGAGATCGAAGAGGAGGTCCGGCAGAAGAAATCATCACGGAAGCGCA
AGCGAGACAGCGACGCCGGCTCCTCCACCCCGACCACCAGCACCCGCAGCCGCGACAAGGACGACGAGAG
CAAGAAGCAGAAGAAGCGCGGGCGGCCGCCTGCCGAGAAACTCTCCCCTAACCCACCCAACCTCACCAAG
AAGATGAAGAAGATTGTGGATGCCGTGATCAAGTACAAGGACAGCAGCAGTGGACGTCAGCTCAGCGAGG
TCTTCATCCAGCTGCCCTCGCGAAAGGAGCTGCCCGAGTACTACGAGCTCATCCGCAAGCCCGTGGACTT
CAAGAAGATAAAGGAGCGCATTCGCAACCACAAGTACCGCAGCCTCAACGACCTAGAGAAGGACGTCATG
CTCCTGTGCCAGAACGCACAGACCTTCAACCTGGAGGGCTCCCTGATCTATGAAGACTCCATCGTCTTGC
AGTCGGTCTTCACCAGCGTGCGGCAGAAAATCGAGAAGGAGGATGACAGTGAAGGCGAGGAGAGTGAGGA
GGAGGAAGAGGGCGAGGAGGAAGGCTCCGAATCCGAATCTCGGTCCGTCAAAGTGAAGATCAAGCTTGGC
CGGAAGGAGAAGGCACAGGACCGGCTGAAGGGCGGCCGGCGGCGGCCGAGCCGAGGGTCCCGAGCCAAGC
CGGTCGTGAGTGACGATGACAGTGAGGAGGAACAAGAGGAGGACCGCTCAGGAAGTGGCAGCGAAGAAGA
CTGAGCCCCGACATTCCAGTCTCGACCCCGAGCCCCTCGTTCCAGAGCTGAGATGGCATAGGCCTTAGCA
GTAACGGGTAGCAGCAGATGTAGTTTCAGACTTGGAGTAAAACTGTATAAACAAAAGAATCTTCCATATT
TATACAGCAGAGAAGCTGTAGGACTGTTTGTGACTGGCCCTGTCCTGGCATCAGTAGCATCTGTAACAGC
ATTAACTGTCTTAAAGAGAGAGAGAGAGAATTCCGAATTGGGGAACACACGATACCTGTTTTTCTTTTCC
GTTGCTGGCAGTACTGTTGCGCCGCAGTTTGGAGTCACTGTAGTTAAGTGTGGATGCATGTGCGTCACCG
TCCACTCCTCCTACTGTATTTTATTGGACAGGTCAGACTCGCCGGGGGCCCGGCGAGGGTATGTCAGTGT
CACTGGATGTCAAACAGTAATAAATTAAACCAACAACAAAACGCACAGCCAAAAAAAAA

The term “BRG1” also refers to natural variants of the wild-type human BRG1 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to an amino acid sequence of wild-type BRG1, which is set forth in SEQ ID NO: 2 (UniProt Accession No.: P51532; www.uniprot.org/uniprot/P51532.fasta).

SEQ ID NO: 2
MSTPDPPLGGTPRPGPSPGPGPSPGAMLGPSPGPSPGSAHSMMGPSPGP
PSAGHPIPTQGPGGYPQDNMHQMHKPMESMHEKGMSDDPRYNQMKGMGM
RSGGHAGMGPPPSPMDQHSQGYPSPLGGSEHASSPVPASGPSSGPQMSS
GPGGAPLDGADPQALGQQNRGPTPFNQNQLHQLRAQIMAYKMLARGQPL
PDHLQMAVQGKRPMPGMQQQMPTLPPPSVSATGPGPGPGPGPGPGPGPA
PPNYSRPHGMGGPNMPPPGPSGVPPGMPGQPPGGPPKPWPEGPMANAAA
PTSTPQKLIPPQPTGRPSPAPPAVPPAASPVMPPQTQSPGQPAQPAPMV
PLHQKQSRITPIQKPRGLDPVEILQEREYRLQARIAHRIQELENLPGSL
AGDLRTKATIELKALRLLNFQRQLRQEVVVCMRRDTALETALNAKAYKR
SKRQSLREARITEKLEKQQKIEQERKRRQKHQEYLNSILQHAKDFKEYH
RSVTGKIQKLTKAVATYHANTEREQKKENERIEKERMRRLMAEDEEGYR
KLIDQKKDKRLAYLLQQTDEYVANLTELVRQHKAAQVAKEKKKKKKKKK
AENAEGQTPAIGPDGEPLDETSQMSDLPVKVIHVESGKILTGTDAPKAG
QLEAWLEMNPGYEVAPRSDSEESGSEEEEEEEEEEQPQAAQPPTLPVEE
KKKIPDPDSDDVSEVDARHIIENAKQDVDDEYGVSQALARGLQSYYAVA
HAVTERVDKQSALMVNGVLKQYQIKGLEWLVSLYNNNLNGILADEMGLG
KTIQTIALITYLMEHKRINGPFLIIVPLSTLSNWAYEFDKWAPSVVKVS
YKGSPAARRAFVPQLRSGKFNVLLTTYEYIIKDKHILAKIRWKYMIVDE
GHRMKNHHCKLTQVLNTHYVAPRRLLLTGTPLQNKLPELWALLNFLLPT
IFKSCSTFEQWFNAPFAMTGEKVDLNEEETILIIRRLHKVLRPFLLRRL
KKEVEAQLPEKVEYVIKCDMSALQRVLYRHMQAKGVLLTDGSEKDKKGK
GGTKTLMNTIMQLRKICNHPYMFQHIEESFSEHLGFTGGIVQGLDLYRA
SGKFELLDRILPKLRATNHKVLLFCQMTSLMTIMEDYFAYRGFKYLRLD
GTTKAEDRGMLLKTFNEPGSEYFIFLLSTRAGGLGLNLQSADTVIIFDS
DWNPHQDLQAQDRAHRIGQQNEVRVLRLCTVNSVEEKILAAAKYKLNVD
QKVIQAGMFDQKSSSHERRAFLQAILEHEEQDESRHCSTGSGSASFAHT
APPPAGVNPDLEEPPLKEEDEVPDDETVNQMIARHEEEFDLFMRMDLDR
RREEARNPKRKPRLMEEDELPSWIIKDDAEVERLTCEEEEEKMFGRGSR
HRKEVDYSDSLTEKQWLKAIEEGTLEEIEEEVRQKKSSRKRKRDSDAGS
STPTTSTRSRDKDDESKKQKKRGRPPAEKLSPNPPNLTKKMKKIVDAVI
KYKDSSSGRQLSEVFIQLPSRKELPEYYELIRKPVDFKKIKERIRNHKY
RSLNDLEKDVMLLCQNAQTFNLEGSLIYEDSIVLQSVFTSVRQKIEKED
DSEGEESEEEEEGEEEGSESESRSVKVKIKLGRKEKAQDRLKGGRRRPS
RGSRAKPVVSDDDSEEEQEEDRSGSGSEED.

As used herein, the term “BRM” refers to probable global transcription activator SNF2L2. BRM is a component of the BAF complex, a SWI/SNF ATPase chromatin remodeling complex. Human BRM is encoded by the SMARCA2 gene on chromosome 9, a nucleic acid sequence of which is set forth in SEQ ID NO: 3. (GenBank Accession No.: NM_003070.4 www.ncbi.nlm.nih.gov/nuccore/NM_003070.4?report=fasta)

SEQ ID NO: 3
GCGTCTTCCGGCGCCCGCGGAGGAGGCGAGGGTGGGACGCTGGGCGGAGCCCGAGTTTAGGAAGAGGAGG
GGACGGCTGTCATCAATGAAGTCATATTCATAATCTAGTCCTCTCTCCCTCTGTTTCTGTACTCTGGGTG
ACTCAGAGAGGGAAGAGATTCAGCCAGCACACTCCTCGCGAGCAAGCATTACTCTACTGACTGGCAGAGA
CAGGAGAGGTAGATGTCCACGCCCACAGACCCTGGTGCGATGCCCCACCCAGGGCCTTCGCCGGGGCCTG
GGCCTTCCCCTGGGCCAATTCTTGGGCCTAGTCCAGGACCAGGACCATCCCCAGGTTCCGTCCACAGCAT
GATGGGGCCAAGTCCTGGACCTCCAAGTGTCTCCCATCCTATGCCGACGATGGGGTCCACAGACTTCCCA
CAGGAAGGCATGCATCAAATGCATAAGCCCATCGATGGTATACATGACAAGGGGATTGTAGAAGACATCC
ATTGTGGATCCATGAAGGGCACTGGTATGCGACCACCTCACCCAGGCATGGGCCCTCCCCAGAGTCCAAT
GGATCAACACAGCCAAGGTTATATGTCACCACACCCATCTCCATTAGGAGCCCCAGAGCACGTCTCCAGC
CCTATGTCTGGAGGAGGCCCAACTCCACCTCAGATGCCACCAAGCCAGCCGGGGGCCCTCATCCCAGGTG
ATCCGCAGGCCATGAGCCAGCCCAACAGAGGTCCCTCACCTTTCAGTCCTGTCCAGCTGCATCAGCTTCG
AGCTCAGATTTTAGCTTATAAAATGCTGGCCCGAGGCCAGCCCCTCCCCGAAACGCTGCAGCTTGCAGTC
CAGGGGAAAAGGACGTTGCCTGGCTTGCAGCAACAACAGCAGCAGCAACAGCAGCAGCAGCAGCAGCAGC
AGCAGCAGCAGCAGCAGCAACAGCAGCCGCAGCAGCAGCCGCCGCAACCACAGACGCAGCAACAACAGCA
GCCGGCCCTTGTTAACTACAACAGACCATCTGGCCCGGGGCCGGAGCTGAGCGGCCCGAGCACCCCGCAG
AAGCTGCCGGTGCCCGCGCCCGGCGGCCGGCCCTCGCCCGCGCCCCCCGCAGCCGCGCAGCCGCCCGCGG
CCGCAGTGCCCGGGCCCTCAGTGCCGCAGCCGGCCCCGGGGCAGCCCTCGCCCGTCCTCCAGCTGCAGCA
GAAGCAGAGCCGCATCAGCCCCATCCAGAAACCGCAAGGCCTGGACCCCGTGGAAATTCTGCAAGAGCGG
GAATACAGACTTCAGGCCCGCATAGCTCATAGGATACAAGAACTGGAAAATCTGCCTGGCTCTTTGCCAC
CAGATTTAAGAACCAAAGCAACCGTGGAACTAAAAGCACTTCGGTTACTCAATTTCCAGCGTCAGCTGAG
ACAGGAGGTGGTGGCCTGCATGCGCAGGGACACGACCCTGGAGACGGCTCTCAACTCCAAAGCATACAAA
CGGAGCAAGCGCCAGACTCTGAGAGAAGCTCGCATGACCGAGAAGCTGGAGAAGCAGCAGAAGATTGAGC
AGGAGAGGAAACGCCGTCAGAAACACCAGGAATACCTGAACAGTATTTTGCAACATGCAAAAGATTTTAA
GGAATATCATCGGTCTGTGGCCGGAAAGATCCAGAAGCTCTCCAAAGCAGTGGCAACTTGGCATGCCAAC
ACTGAAAGAGAGCAGAAGAAGGAGACAGAGCGGATTGAAAAGGAGAGAATGCGGCGACTGATGGCTGAAG
ATGAGGAGGGTTATAGAAAACTGATTGATCAAAAGAAAGACAGGCGTTTAGCTTACCTTTTGCAGCAGAC
CGATGAGTATGTAGCCAATCTGACCAATCTGGTTTGGGAGCACAAGCAAGCCCAGGCAGCCAAAGAGAAG
AAGAAGAGGAGGAGGAGGAAGAAGAAGGCTGAGGAGAATGCAGAGGGTGGGGAGTCTGCCCTGGGACCGG
ATGGAGAGCCCATAGATGAGAGCAGCCAGATGAGTGACCTCCCTGTCAAAGTGACTCACACAGAAACCGG
CAAGGTTCTGTTCGGACCAGAAGCACCCAAAGCAAGTCAGCTGGACGCCTGGCTGGAAATGAATCCTGGT
TATGAAGTTGCCCCTAGATCTGACAGTGAAGAGAGTGATTCTGATTATGAGGAAGAGGATGAGGAAGAAG
AGTCCAGTAGGCAGGAAACCGAAGAGAAAATACTCCTGGATCCAAATAGCGAAGAAGTTTCTGAGAAGGA
TGCTAAGCAGATCATTGAGACAGCTAAGCAAGACGTGGATGATGAATACAGCATGCAGTACAGTGCCAGG
GGCTCCCAGTCCTACTACACCGTGGCTCATGCCATCTCGGAGAGGGTGGAGAAACAGTCTGCCCTCCTAA
TTAATGGGACCCTAAAGCATTACCAGCTCCAGGGCCTGGAATGGATGGTTTCCCTGTATAATAACAACTT
GAACGGAATCTTAGCCGATGAAATGGGGCTTGGAAAGACCATACAGACCATTGCACTCATCACTTATCTG
ATGGAGCACAAAAGACTCAATGGCCCCTATCTCATCATTGTTCCCCTTTCGACTCTATCTAACTGGACAT
ATGAATTTGACAAATGGGCTCCTTCTGTGGTGAAGATTTCTTACAAGGGTACTCCTGCCATGCGTCGCTC
CCTTGTCCCCCAGCTACGGAGTGGCAAATTCAATGTCCTCTTGACTACTTATGAGTATATTATAAAAGAC
AAGCACATTCTTGCAAAGATTCGGTGGAAATACATGATAGTGGACGAAGGCCACCGAATGAAGAATCACC
ACTGCAAGCTGACTCAGGTCTTGAACACTCACTATGTGGCCCCCAGAAGGATCCTCTTGACTGGGACCCC
GCTGCAGAATAAGCTCCCTGAACTCTGGGCCCTCCTCAACTTCCTCCTCCCAACAATTTTTAAGAGCTGC
AGCACATTTGAACAATGGTTCAATGCTCCATTTGCCATGACTGGTGAAAGGGTGGACTTAAATGAAGAAG
AAACTATATTGATCATCAGGCGTCTACATAAGGTGTTAAGACCATTTTTACTAAGGAGACTGAAGAAAGA
AGTTGAATCCCAGCTTCCCGAAAAAGTGGAATATGTGATCAAGTGTGACATGTCAGCTCTGCAGAAGATT
CTGTATCGCCATATGCAAGCCAAGGGGATCCTTCTCACAGATGGTTCTGAGAAAGATAAGAAGGGGAAAG
GAGGTGCTAAGACACTTATGAACACTATTATGCAGTTGAGAAAAATCTGCAACCACCCATATATGTTTCA
GCACATTGAGGAATCCTTTGCTGAACACCTAGGCTATTCAAATGGGGTCATCAATGGGGCTGAACTGTAT
CGGGCCTCAGGGAAGTTTGAGCTGCTTGATCGTATTCTGCCAAAATTGAGAGCGACTAATCACCGAGTGC
TGCTTTTCTGCCAGATGACATCTCTCATGACCATCATGGAGGATTATTTTGCTTTTCGGAACTTCCTTTA
CCTACGCCTTGATGGCACCACCAAGTCTGAAGATCGTGCTGCTTTGCTGAAGAAATTCAATGAACCTGGA
TCCCAGTATTTCATTTTCTTGCTGAGCACAAGAGCTGGTGGCCTGGGCTTAAATCTTCAGGCAGCTGATA
CAGTGGTCATCTTTGACAGCGACTGGAATCCTCATCAGGATCTGCAGGCCCAAGACCGAGCTCACCGCAT
CGGGCAGCAGAACGAGGTCCGGGTACTGAGGCTCTGTACCGTGAACAGCGTGGAGGAAAAGATCCTCGCG
GCCGCAAAATACAAGCTGAACGTGGATCAGAAAGTGATCCAGGCGGGCATGTTTGACCAAAAGTCTTCAA
GCCACGAGCGGAGGGCATTCCTGCAGGCCATCTTGGAGCATGAGGAGGAAAATGAGGAAGAAGATGAAGT
ACCGGACGATGAGACTCTGAACCAAATGATTGCTCGACGAGAAGAAGAATTTGACCTTTTTATGCGGATG
GACATGGACCGGCGGAGGGAAGATGCCCGGAACCCGAAACGGAAGCCCCGTTTAATGGAGGAGGATGAGC
TGCCCTCCTGGATCATTAAGGATGACGCTGAAGTAGAAAGGCTCACCTGTGAAGAAGAGGAGGAGAAAAT
ATTTGGGAGGGGGTCCCGCCAGCGCCGTGACGTGGACTACAGTGACGCCCTCACGGAGAAGCAGTGGCTA
AGGGCCATCGAAGACGGCAATTTGGAGGAAATGGAAGAGGAAGTACGGCTTAAGAAGCGAAAAAGACGAA
GAAATGTGGATAAAGATCCTGCAAAAGAAGATGTGGAAAAAGCTAAGAAGAGAAGAGGCCGCCCTCCCGC
TGAGAAACTGTCACCAAATCCCCCCAAACTGACAAAGCAGATGAACGCTATCATCGATACTGTGATAAAC
TACAAAGATAGGTGTAACGTGGAGAAGGTGCCCAGTAATTCTCAGTTGGAAATAGAAGGAAACAGTTCAG
GGCGACAGCTCAGTGAAGTCTTCATTCAGTTACCTTCAAGGAAAGAATTACCAGAATACTATGAATTAAT
TAGGAAGCCAGTGGATTTCAAAAAAATAAAGGAAAGGATTCGTAATCATAAGTACCGGAGCCTAGGCGAC
CTGGAGAAGGATGTCATGCTTCTCTGTCACAACGCTCAGACGTTCAACCTGGAGGGATCCCAGATCTATG
AAGACTCCATCGTCTTACAGTCAGTGTTTAAGAGTGCCCGGCAGAAAATTGCCAAAGAGGAAGAGAGTGA
GGATGAAAGCAATGAAGAGGAGGAAGAGGAAGATGAAGAAGAGTCAGAGTCCGAGGCAAAATCAGTCAAG
GTGAAAATTAAGCTCAATAAAAAAGATGACAAAGGCCGGGACAAAGGGAAAGGCAAGAAAAGGCCAAATC
GAGGAAAAGCCAAACCTGTAGTGAGCGATTTTGACAGCGATGAGGAGCAGGATGAACGTGAACAGTCAGA
AGGAAGTGGGACGGATGATGAGTGATCAGTATGGACCTTTTTCCTTGGTAGAACTGAATTCCTTCCTCCC
CTGTCTCATTTCTACCCAGTGAGTTCATTTGTCATATAGGCACTGGGTTGTTTCTATATCATCATCGTCT
ATAAACTAGCTTTAGGATAGTGCCAGACAAACATATGATATCATGGTGTAAAAAACACACACATACACAA
ATATTTGTAACATATTGTGACCAAATGGGCCTCAAAGATTCAGATTGAAACAAACAAAAAGCTTTTGATG
GAAAATATGTGGGTGGATAGTATATTTCTATGGGTGGGTCTAATTTGGTAACGGTTTGATTGTGCCTGGT
TTTATCACCTGTTCAGATGAGAAGATTTTTGTCTTTTGTAGCACTGATAACCAGGAGAAGCCATTAAAAG
CCACTGGTTATTTTATTTTTCATCAGGCAATTTTCGAGGTTTTTATTTGTTCGGTATTGTTTTTTTACAC
TGTGGTACATATAAGCAACTTTAATAGGTGATAAATGTACAGTAGTTAGATTTCACCTGCATATACATTT
TTCCATTTTATGCTCTATGATCTGAACAAAAGCTTTTTGAATTGTATAAGATTTATGTCTACTGTAAACA
TTGCTTAATTTTTTTGCTCTTGATTTAAAAAAAAGTTTTGTTGAAAGCGCTATTGAATATTGCAATCTAT
ATAGTGTATTGGATGGCTTCTTTTGTCACCCTGATCTCCTATGTTACCAATGTGTATCGTCTCCTTCTCC
CTAAAGTGTACTTAATCTTTGCTTTCTTTGCACAATGTCTTTGGTTGCAAGTCATAAGCCTGAGGCAAAT
AAAATTCCAGTAATTTCGAAGAATGTGGTGTTGGTGCTTTCCTAATAAAGAAATAATTTAGCTTGACAAA
AAAAAAAAAAAA.

The term “BRM” also refers to natural variants of the wild-type human BRM protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to an amino acid sequence of wild-type BRM, which is set forth in SEQ ID NO: 4. (Uniprot Accession No.: P51531, www.uniprot.org/uniprot/P51531.fasta)

SEQ ID NO: 4
MSTPTDPGAMPHPGPSPGPGPSPGPILGPSPGPGPSPGSVHSMMGPSP
GPPSVSHPMPTMGSTDFPQEGMHQMHKPIDGIHDKGIVEDIHCGSMKG
TGMRPPHPGMGPPQSPMDQHSQGYMSPHPSPLGAPEHVSSPMSGGGPT
PPQMPPSQPGALIPGDPQAMSQPNRGPSPFSPVQLHQLRAQILAYKML
ARGQPLPETLQLAVQGKRTLPGLQQQQQQQQQQQQQQQQQQQQQQQPQ
QQPPQPQTQQQQQPALVNYNRPSGPGPELSGPSTPQKLPVPAPGGRPS
PAPPAAAQPPAAAVPGPSVPQPAPGQPSPVLQLQQKQSRISPIQKPQG
LDPVEILQEREYRLQARIAHRIQELENLPGSLPPDLRTKATVELKALR
LLNFQRQLRQEVVACMRRDTTLETALNSKAYKRSKRQTLREARMTEKL
EKQQKIEQERKRRQKHQEYLNSILQHAKDFKEYHRSVAGKIQKLSKAV
ATWHANTEREQKKETERIEKERMRRLMAEDEEGYRKLIDQKKDRRLAY
LLQQTDEYVANLTNLVWEHKQAQAAKEKKKRRRRKKKAEENAEGGESA
LGPDGEPIDESSQMSDLPVKVTHTETGKVLFGPEAPKASQLDAWLEMN
PGYEVAPRSDSEESDSDYEEEDEEEESSRQETEEKILLDPNSEEVSEK
DAKQIIETAKQDVDDEYSMQYSARGSQSYYTVAHAISERVEKQSALLI
NGTLKHYQLQGLEWMVSLYNNNLNGILADEMGLGKTIQTIALITYLME
HKRLNGPYLIIVPLSTLSNWTYEFDKWAPSVVKISYKGTPAMRRSLVP
QLRSGKFNVLLTTYEYIIKDKHILAKIRWKYMIVDEGHRMKNHHCKLT
QVLNTHYVAPRRILLTGTPLQNKLPELWALLNFLLPTIFKSCSTFEQW
FNAPFAMTGERVDLNEEETILIIRRLHKVLRPFLLRRLKKEVESQLPE
KVEYVIKCDMSALQKILYRHMQAKGILLTDGSEKDKKGKGGAKTLMNT
IMQLRKICNHPYMFQHIEESFAEHLGYSNGVINGAELYRASGKFELLD
RILPKLRATNHRVLLFCQMTSLMTIMEDYFAFRNFLYLRLDGTTKSED
RAALLKKFNEPGSQYFIFLLSTRAGGLGLNLQAADTVVIFDSDWNPHQ
DLQAQDRAHRIGQQNEVRVLRLCTVNSVEEKILAAAKYKLNVDQKVIQ
AGMFDQKSSSHERRAFLQAILEHEEENEEEDEVPDDETLNQMIARREE
EFDLFMRMDMDRRREDARNPKRKPRLMEEDELPSWIIKDDAEVERLTC
EEEEEKIFGRGSRQRRDVDYSDALTEKQWLRAIEDGNLEEMEEEVRLK
KRKRRRNVDKDPAKEDVEKAKKRRGRPPAEKLSPNPPKLTKQMNAIID
TVINYKDRCNVEKVPSNSQLEIEGNSSGRQLSEVFIQLPSRKELPEYY
ELIRKPVDFKKIKERIRNHKYRSLGDLEKDVMLLCHNAQTFNLEGSQI
YEDSIVLQSVFKSARQKIAKEEESEDESNEEEEEEDEEESESEAKSVK
VKIKLNKKDDKGRDKGKGKKRPNRGKAKPVVSDFDSDEEQDEREQSEG
SGTDDE.

As used herein, the term “degrader” refers to a small molecule compound including a degradation moiety, wherein the compound interacts with a protein (e.g., BRG1 and/or BRM) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BRG1 and/or BRM. In one example, the moiety binds to a protease or a ubiquitin ligase that metabolizes the protein, e.g., BRG1 and/or BRM.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

By “modulating the activity of a BAF complex” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference.

By “reducing the activity of BRG1 and/or BRM” is meant decreasing the level of an activity related to a BRG1 and/or BRM, or a related downstream effect. A non-limiting example of inhibition of an activity of BRG1 and/or BRM is decreasing the level of a BAF complex (e.g., GBAF) in a cell. The activity level of BRG1 and/or BRM may be measured using any method known in the art. In some embodiments, an agent which reduces the activity of BRG1 and/or BRM is a small molecule BRG1 and/or BRM inhibitor By “reducing the level of BRG1 and/or BRM” is meant decreasing the level of BRG1 and/or BRM in a cell or subject. The level of BRG1 and/or BRM may be measured using any method known in the art.

As used herein, the term “inhibiting BRG and/or BRM” refers to blocking or reducing the level or activity of the ATPase catalytic binding domain or the bromodomain of the protein. BRG1 and/or BRM inhibition may be determined using methods known in the art, e.g., a BRG and/or BRM ATPase assay, a Nano DSF assay, or a BRG1 and/or BRM Luciferase cell assay.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

As used herein, the term “inhibitor” refers to any agent which reduces the level and/or activity of a protein (e.g., BRG1 and/or BRM). Non-limiting examples of inhibitors include small molecule inhibitors, degraders, antibodies, enzymes, or polynucleotides (e.g., siRNA).

As used herein, the term “LXS196,” refers to the PKC inhibitor having the structure:

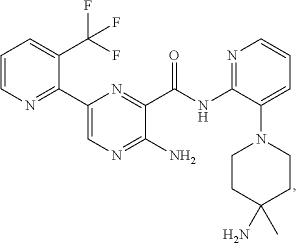 [SMILES: CC1(N)CCN(c2cccnc2NC(=O)c2nc(-c3ncccc3C(F)(F)F)cnc2N)CC1]

or a pharmaceutically acceptable salt thereof.

As used herein, the terms “effective amount,” “therapeutically effective amount,” and “a “sufficient amount” of an agent that reduces the level and/or activity of BRG1 and/or BRM (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating cancer, it is an amount of the agent that reduces the level and/or activity of BRG1 and/or BRM sufficient to achieve a treatment response as compared to the response obtained without administration of the agent that reduces the level and/or activity of BRG1 and/or BRM. The amount of a given agent that reduces the level and/or activity of BRG1 and/or BRM described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of BRG1 and/or BRM of the present disclosure is an amount which results in a beneficial or desired result in a subject as compared to a control. As defined herein, a therapeutically effective amount of an agent that reduces the level and/or activity of BRG1 and/or BRM of the present disclosure may be readily determined by one of ordinary skill by routine methods known in the art. Dosage regimen may be adjusted to provide the optimum therapeutic response.

The term “inhibitory RNA agent” refers to an RNA, or analog thereof, having sufficient sequence complementarity to a target RNA to direct RNA interference. Examples also include a DNA that can be used to make the RNA. RNA interference (RNAi) refers to a sequence-specific or selective process by which a target molecule (e.g., a target gene, protein, or RNA) is down-regulated. Generally, an interfering RNA (“iRNA”) is a double-stranded short-interfering RNA (siRNA), short hairpin RNA (shRNA), or single-stranded micro-RNA (miRNA) that results in catalytic degradation of specific mRNAs, and also can be used to lower or inhibit gene expression.

The terms “short interfering RNA” and “siRNA” (also known as “small interfering RNAs”) refer to an RNA agent, preferably a double-stranded agent, of about 10-50 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., Dicer or a homolog thereof).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

The terms “miRNA” and “microRNA” refer to an RNA agent, preferably a single-stranded agent, of about 10-50 nucleotides in length, preferably between about 15-25 nucleotides in length, which is capable of directing or mediating RNA interference. Naturally-occurring miRNAs are generated from stem-loop precursor RNAs (i.e., pre-miRNAs) by Dicer. The term “Dicer” as used herein, includes Dicer as well as any Dicer ortholog or homolog capable of processing dsRNA structures into siRNAs, miRNAs, siRNA-like or miRNA-like molecules. The term microRNA (“miRNA”) is used interchangeably with the term “small temporal RNA” (“stRNA”) based on the fact that naturally-occurring miRNAs have been found to be expressed in a temporal fashion (e.g., during development).

The term “antisense,” as used herein, refers to a nucleic acid comprising a polynucleotide that is sufficiently complementary to all or a portion of a gene, primary transcript, or processed mRNA, so as to interfere with expression of the endogenous gene (e.g., BRG1 and/or BRM). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other.

The term “antisense nucleic acid” includes single-stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity (e.g., 80%, 85%, 86%, 87%, 88%, 89%, 90%, 91%, 92%, 93%, 94%, 95%, 96%, 97%, 98%, 99%, 99.9% identity, or more) with the targeted polypeptide sequence (e.g., a BRG1 and/or BRM polypeptide sequence). The antisense nucleic acid can be complementary to an entire coding strand, or to only a portion thereof. In some embodiments, an antisense nucleic acid molecule is antisense to a “coding region” of the coding strand of a nucleotide sequence. The term “coding region” refers to the region of the nucleotide sequence comprising codons that are translated into amino acid residues. In some embodiments, the antisense nucleic acid molecule is antisense to a “noncoding region” of the coding strand of a nucleotide sequence. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of mRNA, or can be antisense to only a portion of the coding or noncoding region of an mRNA. For example, the antisense oligonucleotide can be complementary to the region surrounding the translation start site. An antisense oligonucleotide can be, for example, about 5, 10, 15, 20, 25, 30, 35, 40, 45, or 50 nucleotides in length.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By a “reference” is meant any useful reference used to compare protein or mRNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

The details of one or more embodiments of the invention are set forth in the description below. Other features, objects, and advantages of the invention will be apparent from the description and from the claims.

### BRIEF DESCRIPTION OF THE DRAWINGS

FIG. 1 is a graph illustrating inhibition of cell proliferation of several cancer cell lines by a BRG1/BRM inhibitor (compound 17).

FIG. 2A is a graph illustrating inhibition of cell proliferation of uveal melanoma cell line 92-1 by a BRG1/BRM inhibitor (compound 17), a MEK inhibitor (Selumetinib), and a PKC inhibitor (LXS196).

FIG. 2B is a graph illustrating inhibition of cell proliferation of uveal melanoma cell line MP41 by a BRG1/BRM inhibitor (compound 17), a MEK inhibitor (Selumetinib), and PKC inhibitor (LXS196).

FIG. 3 is a graph illustrating inhibition of cell proliferation of several cancer cell lines by a BRG1/BRM inhibitor, compound 18.

FIG. 4 is a graph illustrating the area under the curves (AUCs) calculated from dose-response curves for cancer cell lines treated with a BRG1/BRM inhibitor.

FIG. 5 is a graph illustrating inhibition of cell proliferation of uveal melanoma and non-small cell lung cancer cell lines by a BRG1/BRM inhibitor (compound 18).

FIG. 6A is a graph illustrating inhibition of cell proliferation of uveal melanoma cell line 92-1 by a BRG1/BRM inhibitor (compound 18), a MEK inhibitor (Selumetinib), and a PKC inhibitor (LXS196).

FIG. 6B is a graph illustrating inhibition of cell proliferation of uveal melanoma cell line MP41 by a BRG1/BRM inhibitor (compound 18), a MEK inhibitor (Selumetinib), and a PKC inhibitor (LXS196).

FIG. 7A is a graph illustrating inhibition of cell proliferation of parental and PKC-inhibitor refractory uveal melanoma cell lines by a PKC inhibitor (LXS196).

FIG. 7B is a graph illustrating inhibition of cell proliferation of parental and PKC-inhibitor refractory uveal melanoma cell lines by a BRG1/BRM inhibitor (compound 18).

FIG. 8A is a graph illustrating inhibition of tumor growth in mice engrafted with uveal melanoma cell lines by a BRG1/BRM inhibitor (compound 19).

FIG. 8B is an illustration of the size of tumors from mice engrafted with uveal melanoma cell lines and dosed with a BRG1/BRM inhibitor (compound 19).

FIG. 8C is a graph illustrating body weight change of mice engrafted with uveal melanoma cell lines and dosed with a BRG1/BRM inhibitor (compound 19).

### DETAILED DESCRIPTION

The present inventors have found that depletion of BRG1 and/or BRM in uveal melanoma, prostate cancer, or hematologic cancer cells results in decreased proliferation of the cancer cells.

Accordingly, the invention features methods and compositions useful for the inhibition of the activity of the BRG1 and/or BRM, e.g., for the treatment of cancer such as uveal melanoma, prostate cancer, or hematologic cancer. The invention further features methods and compositions useful for inhibition of the activity of the BRG1 and/or BRM protein, e.g., for the treatment of cancer such as uveal melanoma, prostate cancer, or hematologic cancer, e.g., in a subject in need thereof. Exemplary methods are described herein.

BRG1 and/or BRM-Reducing Agents

Agents described herein that reduce the level and/or activity of BRG1 and/or BRM in a cell may be an antibody, a protein (such as an enzyme), a polynucleotide, or a small molecule compound. The agents reduce the level of an activity related to BRG1 and/or BRM, or a related downstream effect, or reduce the level of BRG1 and/or BRM in a cell or subject.

In some embodiments, the agent that reduces the level and/or activity of BRG1 and/or BRM in a cell is an enzyme, a polynucleotide, or a small molecule compound such as a small molecule BRG1 and/or BRM inhibitor.

Antibodies

The agent that reduces the level and/or activity of BRG1 and/or BRM can be an antibody or antigen binding fragment thereof. For example, an agent that reduces the level and/or activity of BRG1 and/or BRM described herein is an antibody that reduces or blocks the activity and/or function of BRG1 and/or BRM through binding to BRG1 and/or BRM.

The making and use of therapeutic antibodies against a target antigen (e.g., BRG1 and/or BRM) is known in the art. See, for example, the references cited herein above, as well as Zhiqiang An (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

Polynucleotides

In some embodiments, the agent that reduces the level and/or activity of BRG1 and/or BRM is a polynucleotide. In some embodiments, the polynucleotide is an inhibitory RNA molecule, e.g., that acts by way of the RNA interference (RNAi) pathway. An inhibitory RNA molecule can decrease the expression level (e.g., protein level or mRNA level) of BRG1 and/or BRM. For example, an inhibitory RNA molecule includes a short interfering RNA (siRNA), short hairpin RNA (shRNA), and/or a microRNA (miRNA) that targets full-length BRG1 and/or BRM. A siRNA is a double-stranded RNA molecule that typically has a length of about 19-25 base pairs. A shRNA is a RNA molecule including a hairpin turn that decreases expression of target genes via RNAi. A microRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. Degradation is caused by an enzymatic, RNA-induced silencing complex (RISC).

In some embodiments, the agent that reduces the level and/or activity of BRG1 and/or BRM is an antisense nucleic acid. Antisense nucleic acids include antisense RNA (asRNA) and antisense DNA (asDNA) molecules, typically about 10 to 30 nucleotides in length, which recognize polynucleotide target sequences or sequence portions through hydrogen bonding interactions with the nucleotide bases of the target sequence (e.g., BRG1 and/or BRM). The target sequences may be single- or double-stranded RNA, or single- or double-stranded DNA.

In some embodiments, the polynucleotide decreases the level and/or activity of a negative regulator of function or a positive regulator of function. In other embodiments, the polynucleotide decreases the level and/or activity of an inhibitor of a positive regulator of function.

A polynucleotide of the invention can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity. The polynucleotides mentioned above, may also be provided in a specialized form such as liposomes, microspheres, or may be applied to gene therapy, or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase uptake of the nucleic acid. These moieties may be attached to the nucleic acid at the 3′ or 5′ ends and may also be attached through a base, sugar, or intramolecular nucleoside linkage. Other moieties may be capping groups specifically placed at the 3′ or 5′ ends of the nucleic acid to prevent degradation by nucleases such as exonuclease, RNase, etc. Such capping groups include hydroxyl protecting groups known in the art, including glycols such as polyethylene glycol and tetraethylene glycol. The inhibitory action of the polynucleotide can be examined using a cell-line or animal based gene expression system of the present invention in vivo and in vitro. In some embodiments, the polynucleotide decreases the level and/or activity or function of BRG1 and/or BRM. In embodiments, the polynucleotide inhibits expression of BRG1 and/or BRM. In other embodiments, the polynucleotide increases degradation of BRG1 and/or BRM and/or decreases the stability (i.e., half-life) of BRG1 and/or BRM. The polynucleotide can be chemically synthesized or transcribed in vitro.

Inhibitory polynucleotides can be designed by methods well known in the art. siRNA, miRNA, shRNA, and asRNA molecules with homology sufficient to provide sequence specificity required to uniquely degrade any RNA can be designed using programs known in the art, including, but not limited to, those maintained on websites for Thermo Fisher Scientific, the German Cancer Research Center, and The Ohio State University Wexner Medical Center. Systematic testing of several designed species for optimization of the inhibitory polynucleotide sequence can be routinely performed by those skilled in the art. Considerations when designing interfering polynucleotides include, but are not limited to, biophysical, thermodynamic, and structural considerations, base preferences at specific positions in the sense strand, and homology. The making and use of inhibitory therapeutic agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press 2010. Exemplary inhibitory polynucleotides, for use in the methods of the invention, are provided in Table 1, below. In some embodiments, the inhibitory polynucleotides have a nucleic acid sequence with at least 50% (e.g., at least 50%, at least 60%, at least 70%, at least 80%, at least 85%, at least 90%, at least 95%, at least 96%, at least 97%, at least 98%, at least 99%, or 100%) sequence identity to the nucleic acid sequence of an inhibitory polynucleotide in Table 1. In some embodiments, the inhibitory polynucleotides have a nucleic acid sequence with at least 70% sequence identity (e.g., 70%, 75%, 80%, 85%, 86%, 87%, 88%, 89%, 90%, 91%, 92%, 93%, 94%, 95%, 96%, 97%, 98%, 99%, 99.9% identity, or more) to the nucleic acid sequence of an inhibitory polynucleotide in Table 1.

Construction of vectors for expression of polynucleotides for use in the invention may be accomplished using conventional techniques which do not require detailed explanation to one of ordinary skill in the art. For generation of efficient expression vectors, it is necessary to have regulatory sequences that control the expression of the polynucleotide. These regulatory sequences include promoter and enhancer sequences and are influenced by specific cellular factors that interact with these sequences, and are well known in the art.

Gene Editing

In some embodiments, the agent that reduces the level and/or activity of BRG1 and/or BRM is a component of a gene editing system. For example, the agent that reduces the level and/or activity of BRG1 and/or BRM introduces an alteration (e.g., insertion, deletion (e.g., knockout), translocation, inversion, single point mutation, or other mutation) in BRG1 and/or BRM. In some embodiments, the agent that reduces the level and/or activity of BRG1 and/or BRM is a nuclease. Exemplary gene editing systems include the zinc finger nucleases (ZFNs), Transcription Activator-Like Effector-based Nucleases (TALENs), and the clustered regulatory interspaced short palindromic repeat (CRISPR) system. ZFNs, TALENs, and CRISPR-based methods are described, e.g., in Gaj et al., Trends Biotechnol. 31(7):397-405 (2013).

CRISPR refers to a set of (or system including a set of) clustered regularly interspaced short palindromic repeats. A CRISPR system refers to a system derived from CRISPR and Cas (a CRISPR-associated protein) or other nuclease that can be used to silence or mutate a gene described herein. The CRISPR system is a naturally occurring system found in bacterial and archeal genomes. The CRISPR locus is made up of alternating repeat and spacer sequences. In naturally-occurring CRISPR systems, the spacers are typically sequences that are foreign to the bacterium (e.g., plasmid or phage sequences).

The CRISPR system has been modified for use in gene editing (e.g., changing, silencing, and/or enhancing certain genes) in eukaryotes. See, e.g., Wiedenheft et al., Nature 482(7385):331-338 (2012).

For example, such modification of the system includes introducing into a eukaryotic cell a plasmid containing a specifically-designed CRISPR and one or more appropriate Cas proteins. The CRISPR locus is transcribed into RNA and processed by Cas proteins into small RNAs that include a repeat sequence flanked by a spacer. The RNAs serve as guides to direct Cas proteins to silence specific DNA/RNA sequences, depending on the spacer sequence. See, e.g., Horvath et al., Science 327(5962):167-170 (2010); Makarova et al., Biology Direct 1:7 (2006); Pennisi, Science 341(6148):833-836 (2013). In some examples, the CRISPR system includes the Cas9 protein, a nuclease that cuts on both strands of the DNA. See, e.g., Id.

In some embodiments, in a CRISPR system for use described herein, e.g., in accordance with one or more methods described herein, the spacers of the CRISPR are derived from a target gene sequence, e.g., from a BRG1 and/or BRM sequence. In some embodiments, in a CRISPR system for use described herein, e.g., in accordance with one or more methods described herein, the spacers of the CRISPR are derived from a target gene sequence, e.g., from a BRG1 sequence. In some embodiments, in a CRISPR system for use described herein, e.g., in accordance with one or more methods described herein, the spacers of the CRISPR are derived from a target gene sequence, e.g., from a BRM sequence.

In some embodiments, the agent that reduces the level and/or activity of BRG1 and/or BRM includes a guide RNA (gRNA) for use in a CRISPR system for gene editing. Exemplary gRNAs, for use in the methods of the invention, are provided in Table 1, below. In embodiments, the agent that reduces the level and/or activity of BRG1 and/or BRM includes a ZFN, or an mRNA encoding a ZFN, that targets (e.g., cleaves) a nucleic acid sequence (e.g., DNA sequence) of BRG1 and/or BRM. In embodiments, the agent that reduces the level and/or activity of BRG1 and/or BRM includes a TALEN, or an mRNA encoding a TALEN, that targets (e.g., cleaves) a nucleic acid sequence (e.g., DNA sequence) of BRG1 and/or BRM. In embodiments, the agent that reduces the level and/or activity of BRG1 and/or BRM includes a TALEN, or an mRNA encoding a TALEN, that targets (e.g., cleaves) a nucleic acid sequence (e.g., DNA sequence) of BRG1. In embodiments, the agent that reduces the level and/or activity of BRG1 and/or BRM includes a TALEN, or an mRNA encoding a TALEN, that targets (e.g., cleaves) a nucleic acid sequence (e.g., DNA sequence) of BRM.

For example, the gRNA can be used in a CRISPR system to engineer an alteration in a gene (e.g., BRG1 and/or BRM). In other examples, the ZFN and/or TALEN can be used to engineer an alteration in a gene (e.g., BRG1 and/or BRM). Exemplary alterations include insertions, deletions (e.g., knockouts), translocations, inversions, single point mutations, or other mutations. The alteration can be introduced in the gene in a cell, e.g., in vitro, ex vivo, or in vivo. In some embodiments, the alteration decreases the level and/or activity of (e.g., knocks down or knocks out) BRG1 and/or BRM, e.g., the alteration is a negative regulator of function. In yet another example, the alteration corrects a defect (e.g., a mutation causing a defect), in BRG1 and/or BRM. In yet another example, the alteration corrects a defect (e.g., a mutation causing a defect), in BRG1. In yet another example, the alteration corrects a defect (e.g., a mutation causing a defect), in BRM.

In certain embodiments, the CRISPR system is used to edit (e.g., to add or delete a base pair) a target gene, e.g., BRG1 and/or BRM. In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In embodiments, the CRISPR system is used to direct Cas to a promoter of a target gene, e.g., BRG1 and/or BRM, thereby blocking an RNA polymerase sterically. In embodiments, the CRISPR system is used to direct Cas to a promoter of a target gene, e.g., BRG1, thereby blocking an RNA polymerase sterically. In embodiments, the CRISPR system is used to direct Cas to a promoter of a target gene, e.g., BRM, thereby blocking an RNA polymerase sterically.

In some embodiments, a CRISPR system can be generated to edit BRG1 and/or BRM using technology described in, e.g., U.S. Publication No. 20140068797; Cong et al., Science 339(6121):819-823 (2013); Tsai, Nature Biotechnol., 32(6):569-576 (2014); and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., the gene encoding BRG1 and/or BRM. In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some embodiments, CRISPR-mediated gene activation (CRISPRa) can be used for transcriptional activation, e.g., of one or more genes described herein, e.g., a gene that inhibits BRG1 and/or BRM. In the CRISPRa technique, dCas9 fusion proteins recruit transcriptional activators. For example, dCas9 can be used to recruit polypeptides (e.g., activation domains) such as VP64 or the p65 activation domain (p65D) and used with sgRNA (e.g., a single sgRNA or multiple sgRNAs), to activate a gene or genes, e.g., endogenous gene(s). Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1). The CRISPRi and CRISPRa techniques are described in greater detail, e.g., in Dominguez et al., Nat. Rev. Mol. Cell Biol. 17(1):5-15 (2016), incorporated herein by reference.

Small Molecule Compounds

In some embodiments of the invention, the agent that reduces the level and/or activity of BRG1 and/or BRM in a cell is a small molecule compound. In some embodiments, the small molecule compound is a structure of Formula I-III.

In some embodiments, the small molecule BRG1 and/or BRM inhibitor is a compound, or pharmaceutically acceptable salt thereof, having the structure of Formula I:

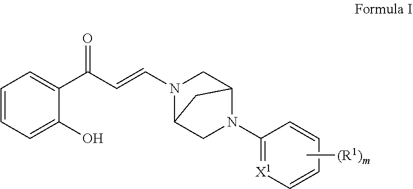

wherein m is 0, 1, 2, 3, or 4;

X¹ is N or CH; and

each R¹ is, independently, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino.

In some embodiments, the small molecule BRG1 and/or BRM inhibitor is a compound, or pharmaceutically acceptable salt thereof, having the structure of Formula II:

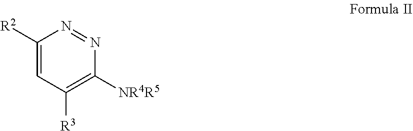

wherein R² is phenyl that is substituted with hydroxy and that is optionally substituted with one or more groups independently selected from the group consisting of halo, cyano, trifluoromethyl, trifluoromethoxy, C₁₋₃ alkyl, and C₁₋₃ alkoxy;

R³ is selected from the group consisting of —R^{a}, —O—R^{a}, —N(R^{a})₂, —S(O)₂R^{a}, and —C(O)—N(R^{a})₂;

each R^{a} is, independently, selected from the group consisting of hydrogen, C₁₋₆ alkyl, C₂₋₆ alkenyl, C₂₋₆ alkynyl, 3-15 membered carbocyclyl, and 3-15 membered heterocyclyl, wherein each C₁₋₆ alkyl, C₂₋₆ alkenyl, C₂₋₆ alkynyl, 3-15 membered carbocyclyl, and 3-15 membered heterocyclyl is optionally substituted with one or more groups independently selected from the group consisting of R^{b}, oxo, halo, —NO₂, —N(R^{b})₂, —CN, —C(O)—N(R^{b})₂, —S(O)—N(R^{b})₂, —S(O)₂—N(R^{b})₂, —O—R^{b}, —S—R^{b}, —O—C(O)—R^{b}, —C(O)—R^{b}, —C(O)—OR^{b}, —S(O)—R^{b}, —S(O)₂—R^{b}, —N(R^{b})—C(O)—R^{b}, —N(R^{b})—S(O)—R^{b}, —N(R^{b})—C(O)—N(R^{b})₂, and —N(R^{b})—S(O)₂—R^{b};

each R^{b} is independently selected from the group consisting of hydrogen, C₁₋₆ alkyl, C₂₋₆ alkenyl, C₂₋₆ alkynyl, C₁₋₆ alkoxy, 3-15 membered carbocyclyl, and 3-15 membered heterocyclyl, wherein each C₁₋₆ alkyl, C₂₋₆ alkenyl, C₂₋₆ alkynyl, C₁₋₆ alkoxy, 3-15 membered carbocyclyl, and 3-15 membered heterocyclyl is optionally substituted with one or more groups independently selected from R^{c}; or two R^{b} are taken together with the nitrogen to which they are attached to form a heterocyclyl that is optionally substituted with one or more groups independently selected from the group consisting of oxo, halo and C₁₋₃ alkyl that is optionally substituted with one or more groups independently selected from the group consisting of oxo and halo;

each R^{c} is independently selected from the group consisting of oxo, halo, —NO₂, —N(R^{d})₂, —CN, —C(O)—N(R^{d})₂, —S(O)—N(R^{d})₂, —S(O)₂—N(R^{d})₂, —S—R^{d}, —O—C(O)—R^{d}, —C(O)—R^{d}, —C(O)—OR^{d}, —S(O)—R^{d}, —S(O)₂—R^{d}, —N(R^{d})—C(O)—R^{d}, —N(R^{d})—S(O)—R^{d}, —N(R^{d})—C(O)—N(R^{d})₂, —N(R^{d})—S(O)₂— R^{d}, C₁₋₆ alkyl, C₂₋₆ alkenyl, C₂₋₆ alkynyl, 3-15 membered carbocyclyl, and 3-15 membered heterocyclyl, wherein any C₁₋₆ alkyl, C₂₋₆ alkenyl, C₂₋₆ alkynyl, 3-15 membered carbocyclyl, and 3-15 membered heterocyclyl is optionally substituted with one or more groups independently selected from the group consisting of R^{d}, oxo, halo, —NO₂, —N(R^{d})₂, —CN, —C(O)—N(R^{d})₂, —S(O)—N(R^{d})₂, —S(O)₂—N(R^{d})₂, —O—R^{d}, —S—R^{d}, —O—C(O)—R^{d}, —C(O)—R^{d}, —C(O)—R^{d}, —S(O)—R^{d}, —S(O)₂—R^{d}, —N(R^{d})—C(O)—R^{d}, —N(R^{d})—S(O)—R^{d}, —N(R^{d})—C(O)—N(R^{d})₂, and —N(R^{d})—S(O)₂—R^{d};

each R^{d} is independently selected from the group consisting of hydrogen, C₁₋₆ alkyl, C₂₋₆ alkenyl, C₂₋₆ alkynyl, carbocyclyl, and carbocyclyl(C₁₋₃ alkyl)-;

R⁴ is H, C₁₋₆ alkyl, or —C(═O)—C₁₋₆ alkyl; and

R⁵ is H or C₁₋₆ alkyl.

Compounds of Formula II may be synthesized by methods known in the art, e.g., those described in U.S. Patent Publication No. 2018/0086720, the synthetic methods of which are incorporated by reference.

In some embodiments, the small molecule BRG1 and/or BRM inhibitor is a compound, or pharmaceutically acceptable salt thereof, having the structure of Formula III:

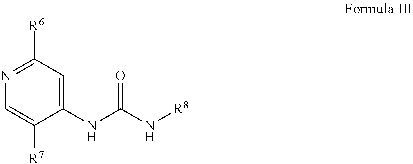

wherein R⁶ is halo, e.g., fluoro or chloro;

R⁷ is hydrogen, optionally substituted amino, or optionally substituted C₁₋₆ alkyl; and

R⁸ is optionally substituted C₆₋₁₀ aryl or optionally substituted C₂₋₉ heteroaryl.

In some embodiments, the small molecule BRG1 and/or BRM inhibitor is a compound, or pharmaceutically acceptable salt thereof, having the structure of any one of compounds 1-16:

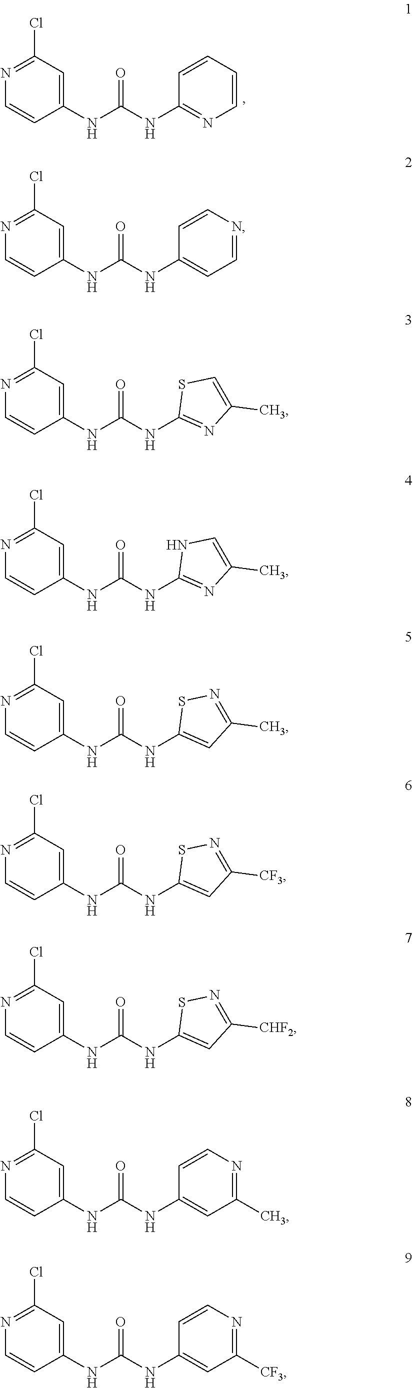

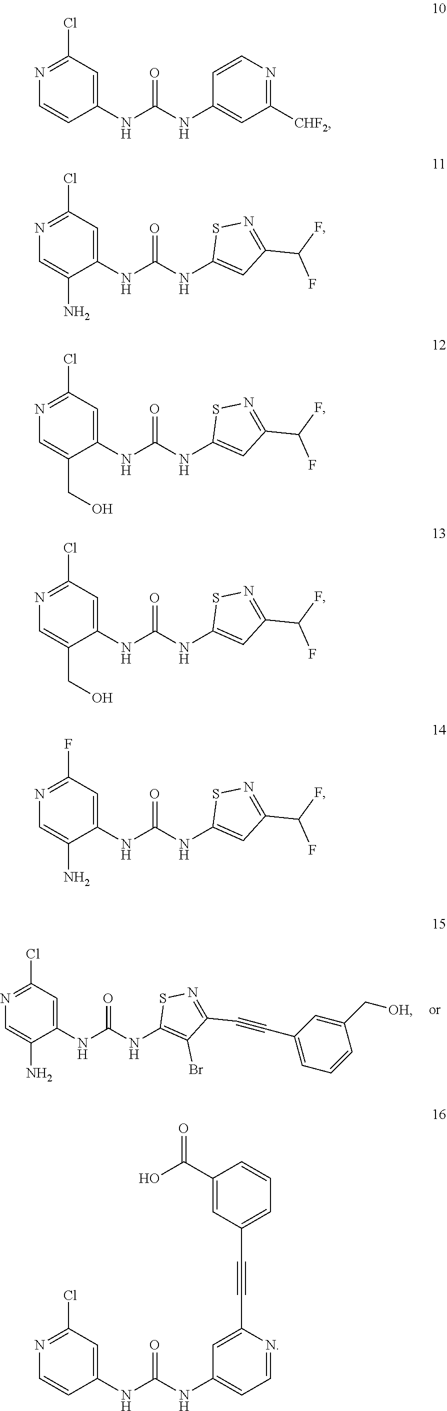

In some embodiments, the small molecule compound, or a pharmaceutically acceptable salt thereof is a degrader. In some embodiments, the degrader has the structure of Formula IV:

A-L-B Formula IV

wherein A is a BRG1 and/or BRM binding moiety; L is a linker; and B is a degradation moiety, or a pharmaceutically acceptable salt thereof. In some embodiments, the degradation moiety is a ubiquitin ligase moiety. In some embodiments, the ubiquitin ligase binding moiety includes Cereblon ligands, IAP (Inhibitors of Apoptosis) ligands, mouse double minute 2 homolog (MDM2), hydrophobic tag, or von Hippel-Lindau ligands, or derivatives or analogs thereof.

In some embodiments, A is a BRG1 binding moiety. In some embodiments, A is a BRM binding moiety. In some embodiments, A includes the structure of any one of Formula I-III, or any one of compounds 1-16.

In some embodiments, the hydrophobic tag includes a diphenylmethane, adamantine, or tri-Boc arginine, i.e., the hydrophobic tag includes the structure:

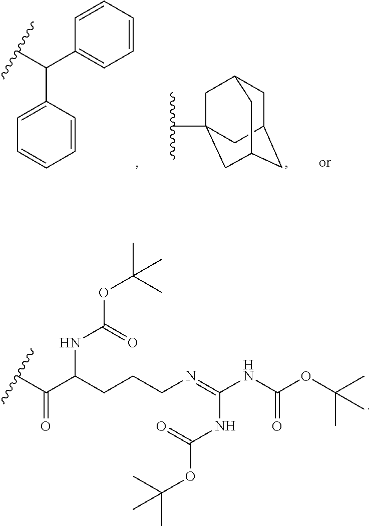 [SMILES: *C(c1ccccc1)c1ccccc1 ; *C12CC3CC(CC(C3)C1)C2 ; *C(=O)C(CCCN=C(NC(=O)OC(C)(C)C)NC(=O)OC(C)(C)C)NC(=O)OC(C)(C)C]

In some embodiments, the ubiquitin ligase binding moiety includes the structure of Formula A:

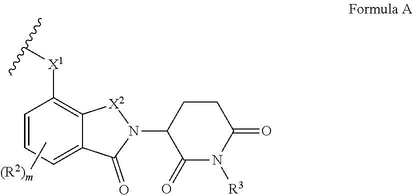

wherein X¹ is CH₂, O, S, or NR¹, wherein R¹ is H, optionally substituted C₁-C₆ alkyl, or optionally substituted C₁-C₆ heteroalkyl; X² is C═O, CH₂, or

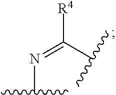

R³ and R⁴ are, independently, H, optionally substituted C₁-C₆ alkyl, or optionally substituted C₁-C₆ heteroalkyl; m is 0, 1, 2, 3, or 4; and each R² is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino,

or a pharmaceutically acceptable salt thereof.

In some embodiments, the ubiquitin ligase binding moiety includes the structure:

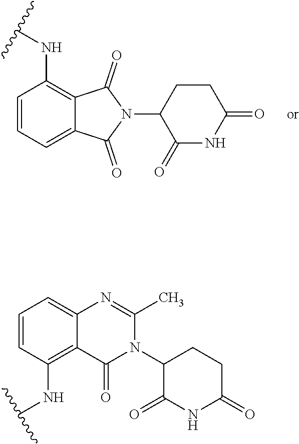 [SMILES: *Nc1cccc2c1C(=O)N(C1CCC(=O)NC1=O)C2=O ; *Nc1cccc2nc(C)n(C3CCC(=O)NC3=O)c(=O)c12]

or is a derivative or an analog thereof, or a pharmaceutically acceptable salt thereof.

In some embodiments, the ubiquitin ligase binding moiety includes the structure of Formula B:

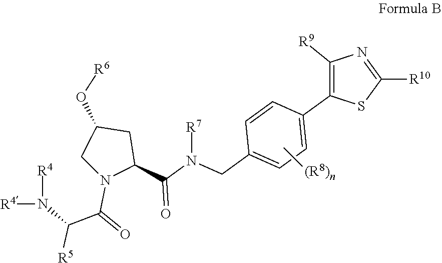

wherein each R⁴, R^{4′}, and R⁷ is, independently, H, optionally substituted C₁-C₆ alkyl, or optionally substituted C₁-C₆ heteroalkyl; R⁵ is optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R⁶ is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
n is 0, 1, 2, 3, or 4; each R⁸ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and each R⁹ and R¹⁰ is, independently, H, halogen, optionally substituted C₁-C₆ alkyl, or optionally substituted C₆-C₁₀ aryl, wherein R^{4′} or R⁵ includes a bond to the linker, or a pharmaceutically acceptable salt thereof.

In some embodiments, the ubiquitin ligase binding moiety includes the structure:

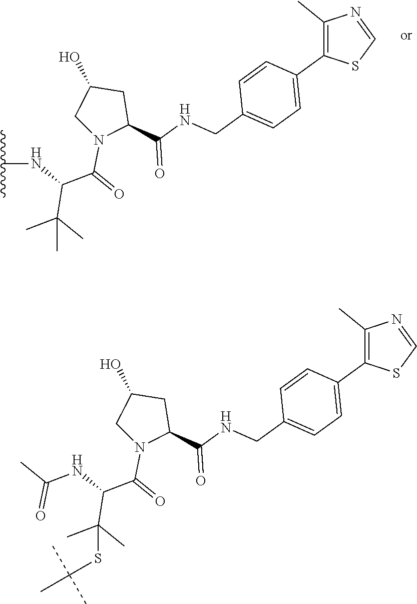

or is a derivative or analog thereof, or a pharmaceutically acceptable salt thereof.

In some embodiments, the ubiquitin ligase binding moiety includes the structure of Formula C:

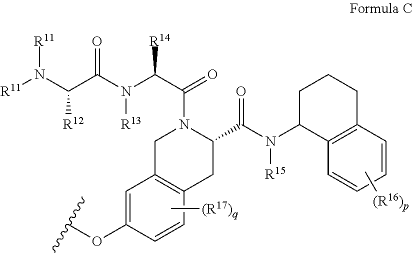

wherein each R¹¹, R¹³, and R¹⁵ is, independently, H, optionally substituted C₁-C₆ alkyl, or optionally substituted C₁-C₆ heteroalkyl; R¹² is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R¹⁴ is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; p is 0, 1, 2, 3, or 4; each R¹⁶ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; q is 0, 1, 2, 3, or 4; and each R¹⁷ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the ubiquitin ligase binding moiety includes the structure:

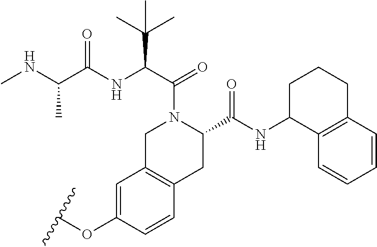 [SMILES: *Oc1ccc2c(c1)CN(C(=O)[C@@H](NC(=O)[C@H](C)NC)C(C)(C)C)[C@H](C(=O)NC1CCCc3ccccc31)C2]

or is a derivative or an analog thereof, or a pharmaceutically acceptable salt thereof.

In some embodiments, the ubiquitin ligase binding moiety includes the structure of Formula D:

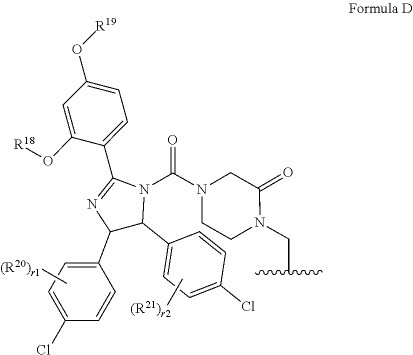

wherein each R¹⁸ and R¹⁹ is, independently, H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; r1 is 0, 1, 2, 3, or 4; each R²⁰ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; r2 is 0, 1, 2, 3, or 4; and each R²¹ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the ubiquitin ligase binding moiety includes the structure:

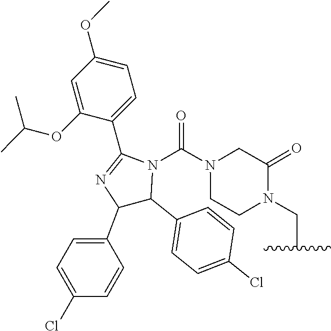 [SMILES: *CN1CCN(C(=O)N2C(c3ccc(OC)cc3OC(C)C)=NC(c3ccc(Cl)cc3)C2c2ccc(Cl)cc2)CC1=O]

or is a derivative or an analog thereof, or a pharmaceutically acceptable salt thereof.

In some embodiments, the linker has the structure of Formula V:

A¹-(B¹)_{f}—(C¹)_{g}—(B²)ₕ-(D)-(B³)ᵢ—(C²)ⱼ—(B⁴)ₖ-A² Formula V

wherein A¹ is a bond between the linker and A; A² is a bond between B and the linker; B¹, B², B³, and B⁴ each, independently, is selected from optionally substituted C₁-C₂ alkyl, optionally substituted C₁-C₃ heteroalkyl, O, S, S(O)₂, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; f, g, h, l, j, and k are each, independently, 0 or 1; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)₁—(B⁴)ₖ-A².

In some embodiments, D is optionally substituted C₂-C₁₀ polyethylene glycol. In some embodiments, C₁ and C² are each, independently, a carbonyl or sulfonyl. In some embodiments, B¹, B², B³, and B⁴ each, independently, is selected from optionally substituted C₁-C₂ alkyl, optionally substituted C₁-C₃ heteroalkyl, 0, S, S(O)₂, and NR^{N}; R^{N} is hydrogen or optionally substituted C₁₋₄ alkyl. In some embodiments, B¹, B², B³, and B⁴ each, independently, is selected from optionally substituted C₁-C₂ alkyl or optionally substituted C₁-C₃ heteroalkyl. In some embodiments, j is 0. In some embodiments, k is 0. In some embodiments, j and k are each, independently, 0. In some embodiments, f, g, h, and i are each, independently, 1.

In some embodiments, the linker of Formula V has the structure of Formula Va:

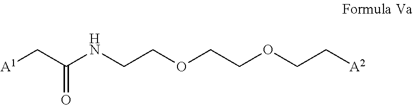

wherein A¹ is a bond between the linker and A, and A² is a bond between B and the linker.

In some embodiments, D is optionally substituted C₁₋₁₀ alkyl. In some embodiments, C₁ and C₂ are each, independently, a carbonyl. In some embodiments, B¹, B², B³, and B⁴ each, independently, is selected from optionally substituted C₁-C₂ alkyl, optionally substituted C₁-C₃ heteroalkyl, 0, S, S(O)₂, and NR^{N}, wherein R^{N} is hydrogen or optionally substituted C₁₋₄ alkyl. In some embodiments, B¹, B², B³, and B⁴ each, independently, is selected from optionally substituted C₁-C₂ alkyl, 0, S, S(O)₂, and NR^{N}, wherein R^{N} is hydrogen or optionally substituted C₁₋₄ alkyl. In some embodiments, B¹ and B⁴ each, independently, is optionally substituted C₁-C₂ alkyl. In some embodiments, B¹ and B⁴ each, independently, is C₁ alkyl. In some embodiments, B² and B⁴ each, independently, is NR^{N}, wherein R^{N} is hydrogen or optionally substituted C₁₋₄ alkyl. In some embodiments, B² and B⁴ each, independently, is NH. In some embodiments, f, g, h, l, j, and k are each, independently, 1.

In some embodiments, the linker of Formula V has the structure of Formula Vb:

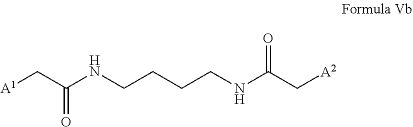

wherein A¹ is a bond between the linker and A, and A² is a bond between B and the linker.

### Pharmaceutical Uses

The compounds described herein are useful in the methods of the invention and, while not bound by theory, are believed to exert their desirable effects through their ability to modulate the level, status, and/or activity of a BAF complex, e.g., by inhibiting the activity or level of the BRG1 and/or BRM proteins in a cell within the BAF complex in a mammal.

An aspect of the present invention relates to methods of treating disorders related to BRG1 and/or BRM proteins such as cancer in a subject in need thereof. In some embodiments, the compound is administered in an amount and for a time effective to result in one of (or more, e.g., two or more, three or more, four or more of): (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, and (i) increased progression free survival of a subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site (e.g., in the liver). For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in inhibition or slowing of the metastatic progression of the cancer. For example, a patient may be administered an amount of an agent that reduces the activity or level of the BRG1 and/or BRM that is effective to inhibit metastasis of the cancer to other parts of the body (e.g., a patient having uveal melanoma that has metastasized (e.g., to the liver)). An agent may be administered in an adjuvant or neo-adjuvant setting, such as prior to or subsequent to surgical rescission of the cancer, and result in a decrease incidence of metastasis of the cancer.

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound described herein. An increase in average survival time of a population may also be measured, for example, by calculating for a population the average length of survival following completion of a first round of treatment with a pharmaceutically acceptable salt of a compound described herein.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of a compound described herein. A decrease in the mortality rate of a population may also be measured, for example, by calculating for a population the average number of disease-related deaths per unit time following completion of a first round of treatment with a pharmaceutically acceptable salt of a compound described herein.

### Combination Therapies

A method of the invention can be used alone or in combination with an additional therapeutic agent, e.g., other agents that treat cancer or symptoms associated therewith, or in combination with other types of therapies to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6 (2005)). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE®, cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al., Proc. Am. Soc. Clin. Oncol. 18:233a (1999), and Douillard et al., Lancet 355(9209):1041-1047 (2000).

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab-I-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOLIRIS® (eculizumab); CIMZIA® (certolizumab pegol); SIMPONI® (golimumab); ILARIS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

In some embodiments, the second agent is dacarbazine, temozolomide, cisplatin, treosulfan, fotemustine, IMCgp100, a CTLA-4 inhibitor (e.g., ipilimumab), a PD-1 inhibitor (e.g., Nivolumab or pembrolizumab), a PD-L1 inhibitor (e.g., atezolizumab, avelumab, or durvalumab), a mitogen-activated protein kinase (MEK) inhibitor (e.g., selumetinib, binimetinib, or tametinib), and/or a protein kinase C (PKC) inhibitor (e.g., sotrastaurin or LXS196).

In some embodiments, the second agent is a mitogen-activated protein kinase (MEK) inhibitor (e.g., selumetinib, binimetinib, or tametinib) and/or a protein kinase C (PKC) inhibitor (e.g., sotrastaurin or LXS196).

The second agent may be a therapeutic agent which is a non-drug treatment. For example, the second therapeutic agent is radiation therapy, thermotherapy, photocoagulation, cryotherapy, hyperthermia, and/or surgical excision of tumor.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody or fusion a protein such as ipilimumab/YERVOY® or tremelimumab). In some embodiments, the inhibitor of checkpoint is an inhibitor (e.g., an inhibitory antibody or small molecule inhibitor) of PD-1 (e.g., nivolumab/OPDIVO®; pembrolizumab/KEYTRUDA®; pidilizumab/CT-011). In some embodiments, the inhibitor of checkpoint is an inhibitor (e.g., an inhibitory antibody or small molecule inhibitor) of PDL1 (e.g., atezolizumab, avelumab, durvalumab, MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/lg fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In some embodiments, the anti-cancer therapy is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

Delivery of anti-BRG1 and/or BRM Agents

A variety of methods are available for the delivery of anti-BRG1 and/or BRM agents to a subject including viral and non-viral methods.

Viral Delivery Methods

In some embodiments, the agent that reduces the level and/or activity of BRG1 and/or BRM is delivered by a viral vector (e.g., a viral vector expressing an anti-BRG1 and/or BRM agent). Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double-stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference.

Exemplary viral vectors include lentiviral vectors, AAVs, and retroviral vectors. Lentiviral vectors and AAVs can integrate into the genome without cell divisions, and both types have been tested in pre-clinical animal studies. Methods for preparation of AAVs are described in the art e.g., in U.S. Pat. Nos. 5,677,158, 6,309,634, and 6,683,058, each of which is incorporated herein by reference. Methods for preparation and in vivo administration of lentiviruses are described in US 20020037281 (incorporated herein by reference). Preferably, a lentiviral vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding the fusion protein, an origin of second strand DNA synthesis and a 3′ lentiviral LTR.

Retroviruses are most commonly used in human clinical trials, as they carry 7-8 kb, and have the ability to infect cells and have their genetic material stably integrated into the host cell with high efficiency (see, e.g., WO 95/30761; WO 95/24929, each of which is incorporated herein by reference). Preferably, a retroviral vector is replication defective. This prevents further generation of infectious retroviral particles in the target tissue. Thus, the replication defective virus becomes a “captive” transgene stable incorporated into the target cell genome. This is typically accomplished by deleting the gag, env, and pol genes (along with most of the rest of the viral genome). Heterologous nucleic acids are inserted in place of the deleted viral genes. The heterologous genes may be under the control of the endogenous heterologous promoter, another heterologous promoter active in the target cell, or the retroviral 5′ LTR (the viral LTR is active in diverse tissues).

These delivery vectors described herein can be made target-specific by attaching, for example, a sugar, a glycolipid, or a protein (e.g., an antibody to a target cell receptor).

Reversible delivery expression systems may also be used. The Cre-loxP or FLP/FRT system and other similar systems can be used for reversible delivery-expression of one or more of the above-described nucleic acids. See WO2005/112620, WO2005/039643, US20050130919, US20030022375, US20020022018, US20030027335, and US20040216178. In particular, the reversible delivery-expression system described in US20100284990 can be used to provide a selective or emergency shut-off.

Non-Viral Delivery Methods

Several non-viral methods exist for delivery of anti-BRG1 and/or BRM agents including polymeric, biodegradable microparticle, or microcapsule delivery devices known in the art. For example, a colloidal dispersion system may be used for targeted delivery an anti-BRG1 and/or BRM agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules.

The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Lipids useful in liposome production include phosphatidyl compounds, such as phosphatidylglycerol, phosphatidylcholine, phosphatidylserine, phosphatidyl-ethanolamine, sphingolipids, cerebrosides, and gangliosides. Exemplary phospholipids include egg phosphatidylcholine, dipalmitoylphosphatidylcholine, and distearoyl-phosphatidylcholine. The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255.

### Pharmaceutical Compositions

The pharmaceutical compositions described herein are preferably formulated into pharmaceutical compositions for administration to human subjects in a biologically compatible form suitable for administration in vivo.

The compounds described herein may be used in the form of the free base, in the form of salts, solvates, and as prodrugs. All forms are within the methods described herein. In accordance with the methods of the invention, the described compounds or salts, solvates, or prodrugs thereof may be administered to a patient in a variety of forms depending on the selected route of administration, as will be understood by those skilled in the art. The compounds described herein may be administered, for example, by oral, parenteral, buccal, sublingual, nasal, rectal, patch, pump, intratumoral, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound described herein may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound described herein may be incorporated with an excipient and used in the form of ingestible tablets, buccal tablets, troches, capsules, elixirs, suspensions, syrups, and wafers. A compound described herein may also be administered parenterally. Solutions of a compound described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature.

The compounds described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds described herein, and/or compositions including a compound described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds described herein may be administered initially in a suitable dosage that may be adjusted as required, depending on the clinical response. In general, satisfactory results may be obtained when the compounds described herein are administered to a human at a daily dosage of, for example, between 0.05 mg and 3000 mg (measured as the solid form). Dose ranges include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

Alternatively, the dosage amount can be calculated using the body weight of the patient. For example, the dose of a compound, or pharmaceutical composition thereof, administered to a patient may range from 0.1-50 mg/kg (e.g., 0.25-25 mg/kg). In exemplary, non-limiting embodiments, the dose may range from 0.5-5.0 mg/kg (e.g., 0.5, 1.0, 1.5, 2.0, 2.5, 3.0, 3.5, 4.0, 4.5, or 5.0 mg/kg) or from 5.0-20 mg/kg (e.g., 5.5, 6.0, 6.5, 7.0, 7.5, 8.0, 8.5, 9.0, 9.5, 10, 11, 12, 13, 14, 15, 16, 17, 18, 19, or 20 mg/kg).

### Kits

The invention also features kits including (a) a pharmaceutical composition including an agent that reduces the level and/or activity of BRG1 and/or BRM in a cell or subject described herein, and (b) a package insert with instructions to perform any of the methods described herein. In some embodiments, the kit includes (a) a pharmaceutical composition including an agent that reduces the level and/or activity of BRG1 and/or BRM in a cell or subject described herein, (b) an additional therapeutic agent (e.g., an anti-cancer agent), and (c) a package insert with instructions to perform any of the methods described herein.

### EXAMPLES

### Example 1. Compound 17

Synthesis of compound 17: BRG1/BRM inhibitor compound 17 has the structure:

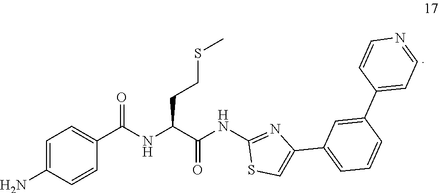 [SMILES: CSCC[C@H](NC(=O)c1ccc(N)cc1)C(=O)Nc1nc(-c2cccc(-c3ccncc3)c2)cs1]

Compound 17 was synthesized as shown in Scheme 1 below.

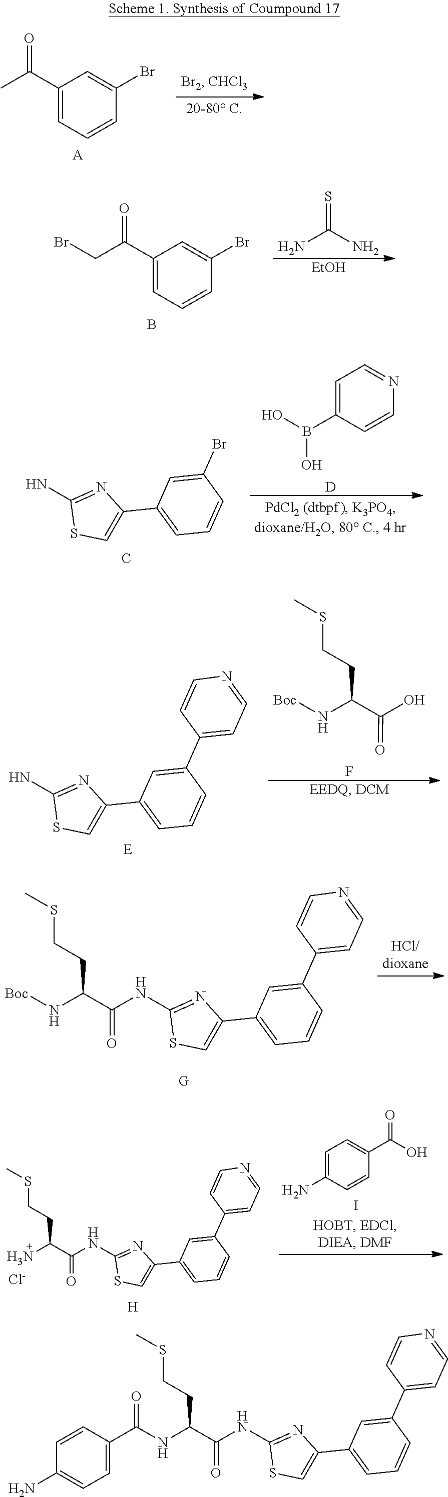

### Step 1: Preparation of 2-bromo-1-(3-bromophenyl)ethenone (Intermediate B)

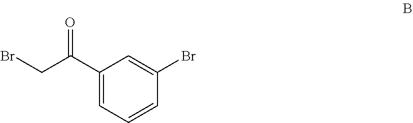 [SMILES: O=C(CBr)c1cccc(Br)c1]

To a solution of 1-(3-bromophenyl)ethanone (132.45 mL, 1.00 mol) in CHCl₃ (250 mL) was added Br₂ (77.70 mL, 1.51 mol) in a dropwise manner at 20° C. under N2 (g). The reaction mixture was subsequently stirred at 80° C. After 1 h, the mixture was cooled to room temperature and concentrated to give intermediate B (279.27 g) as yellow oil, which was used for next step directly.

### Step 2: Preparation of 4-(3-bromophenyl)thiazol-2-amine (Intermediate C)

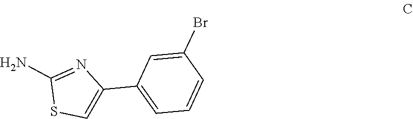 [SMILES: Nc1nc(-c2cccc(Br)c2)cs1]

To a solution of thiourea (229.23 g, 3.01 mol) in EtOH (1.5 L) was added intermediate B (279 g, 1.00 mol). The reaction mixture was stirred at 85° C. After 2 h, the mixture was cooled to room temperature and concentrated in vacuum to give an oil. The oil was carefully poured into saturated aqueous NaHCO₃ (2 L). The resulting basic (pH ˜8) solution was extracted with ethyl acetate (3×2 L). The combined organic layers were washed with brine (4 L), dried over Na₂SO₄, filtered, and concentrated to give an oil. The oil was purified by column chromatography (SiO₂. PE:EA=5:1) to give intermediate C (130.00 g, 494.40 mmol, 49.25% yield) as a yellow solid. LCMS (ESI) m/z: [M+H]⁺=257.0. ¹H NMR (400 MHz, DMSO-d6) δ 7.98 (m, 1H), 7.79 (d, J=8.0 Hz, 1H), 7.44-7.42 (m, 1H), 7.33-7.29 (m, 1H), 7.14 (s, 1H), 7.09 (s, 2H).

### Step 3: Preparation of 4-[3-(4-pyridyl)phenyl]thiazol-2-amine (Intermediate E)

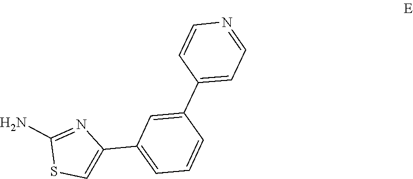 [SMILES: Nc1nc(-c2cccc(-c3ccncc3)c2)cs1]

Intermediate C (20.00 g, 78.40 mmol), 4-pyridylboronic acid (28.9 g, 239.18 mmol), dichloro[1,1′-bis(di-t-butylphosphino)ferrocene]palladium(II) (2.56 g, 3.92 mmol) and K₃PO₄ (66.56 g, 313.56 mmol) were diluted in 1,4-dioxane (240 mL) and water (24 mL). The mixture was purged with N2 (g) three times and then stirred at 80° C. After 7 h, the reaction mixture was cooled to room temperature and water (800 mL) was added. The mixture was extracted with EtOAc (3×800 mL). The combined organic layers were washed with brine, dried over anhydrous Na₂SO₄, filtered, and concentrated in vacuo. The resulting oil was stirred over a mixture of dichloromethane (30 mL) and MTBE (100 mL). After stirring for 5 min, the precipitate was filtered and washed with MTBE (10 mL) to give intermediate E (16.20 g, 61.17 mmol, 78.03% yield) as a yellow solid. LCMS (ESI) m/z: [M+H]⁺=254.0.

### Step 4: Preparation (S)-4-(methylthio)-1-oxo-1-((4-(3-(pyridin-4-yl)phenyl)thiazol-2-yl)amino)butan-2-aminium chloride (Intermediate G)

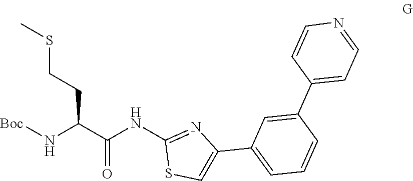 [SMILES: CSCC[C@H](NC(=O)OC(C)(C)C)C(=O)Nc1nc(-c2cccc(-c3ccncc3)c2)cs1]

To a mixture of intermediate E (12.60 g, 49.74 mmol) and (2S)-2-(tert-butoxycarbonylamino)-4-methylsulfanyl-butanoic acid (18.60 g, 74.61 mmol) in dichloromethane (900 mL) was added EEDQ (24.60 g, 99.48 mmol). After stirring for 2 h at room temperature, the reaction mixture was concentrated in vacuo. The residue was triturated with dichloromethane (100 mL) followed by MeOH (200 mL) to give the intermediate G (11.70 g, 23.73 mmol, 47.71% yield, ee %=99.44%) as white solids. LCMS (ESI) m/z: [M+H]⁺=485.1. ¹H NMR (400 MHz, DMSO) δ 12.39 (s, 1H), 8.68-8.66 (m, 2H), 8.30 (s, 1H), 8.02-7.99 (m, 1H), 7.83 (s, 1H), 7.76-7.74 (m, 3H), 7.61-7.57 (m, 1H), 7.28 (d, J=7.6 Hz, 1H), 4.31-4.30 (m, 1H), 2.65-2.44 (m, 2H), 2.06 (s, 3H) 2.01-1.85 (m, 2H), 1.38 (s, 9H).

### Step 5: Preparation of (S)-4-(methylthio)-1-oxo-1-((4-(3-(pyridin-4-yl)phenyl)thiazol-2-yl)amino)butan-2-aminium chloride (Intermediate H)

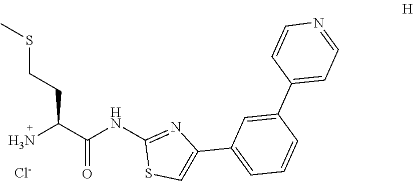 [SMILES: CSCC[C@H](N)C(=O)Nc1nc(-c2cccc(-c3ccncc3)c2)cs1 ; [Cl-]]

A mixture of intermediate G (11.50 g, 23.73 mmol) in MeOH (50 mL) was added a solution of 4 M HCl in 1,4-dioxane (100 mL). After stirring for 1 h at room temperature, the mixture was poured into MTBE (1000 mL). The resulting precipitates were filtered to give the intermediate H (9.99 g, 23.73 mmol, 100.00% yield, HCl salt) as a yellow solid. LCMS (ESI) m/z: [M+H]⁺=385.0

### Step 6: Preparation of 4-amino-N-[(1S)-3-methylsulfanyl-1-[[4-[3-(4-pyridyl)phenyl]thiazol-2-yl]carbamoyl]propyl]benzamide (compound 17)

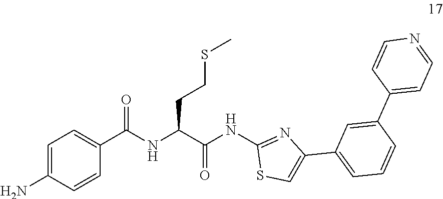 [SMILES: CSCC[C@H](NC(=O)c1ccc(N)cc1)C(=O)Nc1nc(-c2cccc(-c3ccncc3)c2)cs1]

To a mixture of intermediate H (4.00 g, 9.50 mmol) and 4-aminobenzoic acid (1.30 g, 9.50 mmol) in DMF (40 mL) was sequentially added N,N-diisopropylethylamine (6.62 mL, 38.01 mmol), EDCI (2.73 g, 14.25 mmol) and HOBt (1.93 g, 14.25 mmol). The solution was stirred at 25° C. for 14 h and subsequently poured into water (200 mL). The resulting precipitates were collected by filtration. The solids were triturated in MeOH (200 mL) and filtered The solids were further purified by column chromatography (SiO₂, DCM:MeOH=80:1-20:1) to give compound 17 (2.13 g, 4.19 mmol, 44.11% yield, ee %=99.28%) as white solids. LCMS (ESI) m/z: [M+H]⁺=504.0. ¹H NMR (400 MHz, DMSO) δ 12.40 (s, 1H), 8.68-8.66 (m, 2H), 8.31-8.30 (m, 1H), 8.22 (d, J=7.2 Hz, 1H), 8.02-7.99 (m, 1H), 7.82 (s, 1H), 7.76-7.74 (m, 3H), 7.67-7.63 (m, 2H), 7.61-7.57 (m, 1H), 6.58-6.54 (m, 2H), 5.67 (s, 2H), 4.72-4.67 (m, 1H), 2.65-2.54 (m, 2H), 2.12-2.06 (m, 5H).

ATPase activity of compound 17: The ATPase catalytic activity of BRM or BRG-1 in the presence of compound 17 was measured by the in vitro biochemical assay using ADP-Glo™ (Promega, V9102). The ADP-Glo™ kinase assay is performed in two steps once the reaction is complete. The first step is to deplete any unconsumed ATP in the reaction. The second step is to convert the reaction product ADP to ATP, which will be utilized by the luciferase to generate luminesce and be detected by a luminescence reader, such as Envision.

The assay reaction mixture (10 μL) contains 30 nM of BRM or BRG1, 20 nM salmon sperm DNA (from Invitrogen, UltraPure™ Salmon Sperm DNA Solution, cat #15632011), and 400 μM of ATP in the ATPase assay buffer, which comprises of 20 mM Tris, pH 8, 20 mM MgCl₂, 50 mM NaCl, 0.1% Tween-20, and 1 mM fresh DTT (Pierce™ DTT (Dithiothreitol), cat #20290). The reaction is initiated by the addition of the 2.5 μL ATPase solution to 2.5 μL ATP/DNA solution on low volume white Proxiplate-384 plus plate (PerkinElmer, cat #6008280) and incubates at room temperature for 1 hour. Then following addition of 5 μL of ADP-Glo™ Reagent provided in the kit, the reaction incubates at room temperature for 40 minutes. Then 10 μL of Kinase Detection Reagent provided in the kit is added to convert ADP to ATP, and the reaction incubates at room temperature for 60 minutes. Finally, luminescence measurement is collected with a plate-reading luminometer, such as Envision.

BRM and BRG1 were synthesized from high five insect cell lines with a purity of greater than 90%. Compound 17 was found to have an IP₅₀ of 10.4 nM against BRM and 19.3 nM against BRG1 in the assay.

### Example 2. Effects of BRG1/BRM ATPase Inhibition on the Growth of Uveal Melanoma and Hematological Cancer Cell Lines

Procedure: Uveal melanoma cell lines (92-1, MP41, MP38, MP46), prostate cancer cell lines (LNCAP), lung cancer cell lines (NCIH1299), and immortalized embryonic kidney lines (HEK293T) were plated into 96 well plates with growth media (see Table 1). BRG1/BRM ATPase inhibitor, compound 17, was dissolved in DMSO and added to the cells in a concentration gradient from 0 to 10 micromolar at the time of plating. Cells were incubated at 37° C. for 3 days. After 3 days of treatment, the media was removed from the cells, and 30 microliters of TrypLE (Gibco) was added to cells for 10 minutes. Cells were detached from the plates and resuspended with the addition of 170 microliters of growth media. Cells from two DMSO-treated control wells were counted, and the initial number of cells plated at the start of the experiment, were re-plated into fresh-compound containing plates for an additional four days at 37° C. At day 7, cells were harvested as described above.

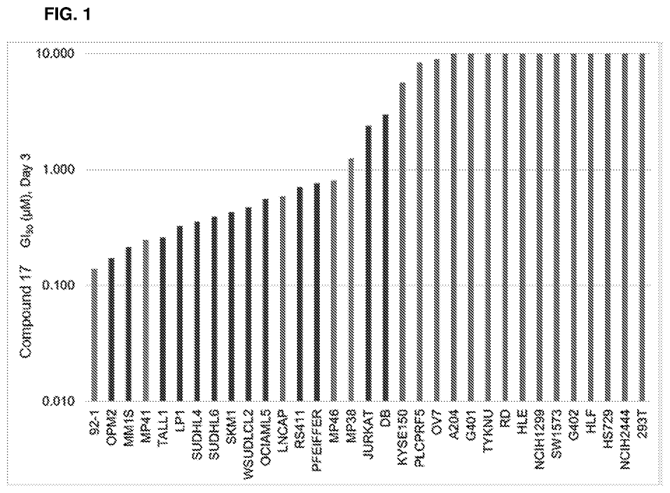

On day 3 and day 7, relative cell growth was measured by the addition of Cell-titer glo (Promega), and luminescence was measured on an Envision plate reader (Perkin Elmer). The concentration of compound 17 at which each cell line's growth was inhibited by 50% (Glso) was calculated using Graphpad Prism and is plotted in FIG. 1.

For multiple myeloma cell lines (OPM2, MM1 S, LP1), ALL cell lines (TALL1, JURKAT, RS411), DLBCL cell lines (SUDHL6, SUDHL4, DB, WSUDLCL2, PFEIFFER), AML cell lines (OCIAML5), MDS cell lines (SKM1), ovarian cancer cell lines (OV7, TYKNU), esophageal cancer cell lines (KYSE150), rhabdoid tumor lines (RD, G402, G401, HS729, A204), liver cancer cell lines (HLF, HLE, PLCRPF5), and lung cancer cell lines (SW1573, NCIH2444), the above methods were performed with the following modifications: Cells were plated in 96 well plates, and the next day, BRG1/BRM ATPase inhibitor, compound 17, was dissolved in DMSO and added to the cells in a concentration gradient from 0 to 10 micromolar. At the time of cell splitting on days 3 and 7, cells were split into new 96 well plates, and fresh compound was added four hours after re-plating.

Table 1 lists the tested cell lines and growth media used.

TABLE 1
Cell Lines and Growth Media

| Cell Line | Source | Growth Media |
|---|---|---|
| 92-1 | SIGMA | RPMI1640 + 20% FBS |
| A204 | ATCC | McCoy's 5A + 10% FBS |
| DB | ATCC | RPMI1640 + 10% FBS |
| G401 | ATCC | McCoy's 5A + 10% FBS |
| G402 | ATCC | McCoy's 5A + 10% FBS |
| HEK293T | ATCC | DMEM + 10% FBS |
| HLE | JCRB | DMEM + 10% FBS |
| HLF | JCRB | DMEM + 10% FBS |
| HS729 | ATCC | DMEM + 10% FBS |
| JURKAT | ATCC | RPMI1640 + 10% FBS |
| KYSE150 | DSMZ | RPMI1640/Ham's F12 + |
| | | 10% FBS |
| LNCAP | ATCC | RPMI1640 + 10% FBS |
| LP1 | DSMZ | IMDM + 20% FBS |
| MM1S | ATCC | RPMI1640 + 10% FBS |
| MP38 | ATCC | RPMI1640 + 20% FBS |
| MP41 | ATCC | RPMI1640 + 20% FBS |
| MP46 | ATCC | RPMI1640 + 20% FBS |
| NCIH1299 | ATCC | RPMI1640 + 10% FBS |
| NCIH2444 | ATCC | RPMI1640 + 20% FBS |
| OCIAML5 | DSMZ | alpha-MEM + 20% FBS + |
| | | 10 ng/ml GM-CSF |
| OPM2 | DSMZ | RPMI1640 + 10% FBS |
| OV7 | ECACC | DMEM/Ham's F12 (1:1) + |
| | | 2 mM Glutamine + 10% FBS + |
| | | 0.5 ug/ml hydrocortisone + |
| | | 10 ug/ml insulin |
| PFEIFFER | ATCC | RPMI1640 + 10% FBS |
| PLCPRF5 | ATCC | EMEM + 10% FBS |
| RD | ATCC | DMEM + 10% FBS |
| RS411 | ATCC | RPMI1640 + 10% FBS |
| SKM1 | JCRB | RPMI1640 + 10% FBS |
| SUDHL4 | DSMZ | RPMI1640 + 10% FBS |
| SUDHL6 | ATCC | RPMI1640 + 20% FBS |
| SW1573 | ATCC | DMEM + 10% FBS |
| TALL1 | JCRB | RPMI1640 + 10% FBS |
| TYKNU | JCRB | EMEM + 20% FBS |
| WSUDLCL2 | DSMZ | RPMI1640 + 10% FBS |

Results: As shown in FIG. 1, the uveal melanoma and hematologic cancer cell lines were more sensitive to BRG1/BRM inhibition than the other tested cell lines. Inhibition of the uveal melanoma and hematologic cancer cell lines was maintained through day 7.

### Example 3. Comparison of BRG1/BRM Inhibitors to Clinical PKC and MEK Inhibitors in Uveal Melanoma Cell Lines

Procedure: Uveal melanoma cell lines, 92-1 or MP41, were plated in 96 well plates in the presence of growth media (see Table 1). BAF ATPase inhibitor (compound 17), PKC inhibitor (LXS196; MedChemExpress), and MEK inhibitor (Selumetinib; Selleck Chemicals) were dissolved in DMSO and added to the cells in a concentration gradient from 0 to 10 micromolar at the time of plating. Cells were incubated at 37° C. for 3 days. After 3 days of treatment, cell growth was measured with Cell-titer glow (Promega), and luminescence was read on an Envision plate reader (Perkin Elmer).

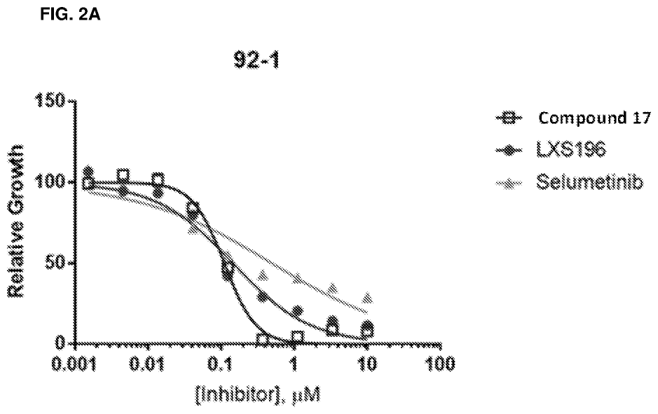

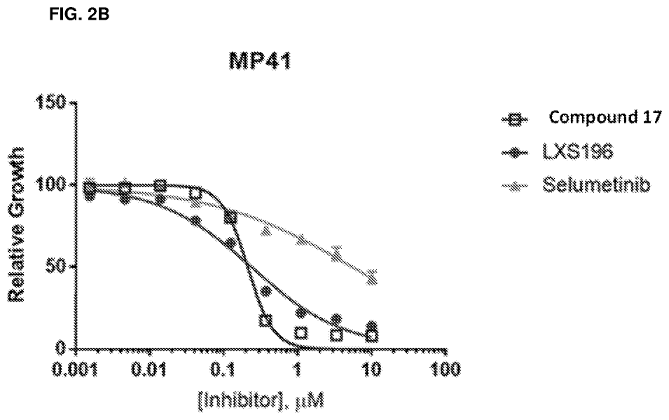

Results: As shown in FIG. 2A and FIG. 21B, compound 17 showed comparable growth inhibition of uveal melanoma cells as the clinical PKC and MEK inhibitors. Further, compound 17 was found to result in a faster onset of inhibition than the clinical PKC and MEK inhibitors.

### Example 4. Synthesis of Compound 18

BRG1/BRM Inhibitor compound 18 has the structure:

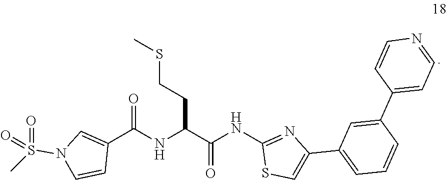 [SMILES: CSCC[C@H](NC(=O)c1ccn(S(C)(=O)=O)c1)C(=O)Nc1nc(-c2cccc(-c3ccncc3)c2)cs1]

Compound 18 was synthesized as shown in Scheme 2 below.

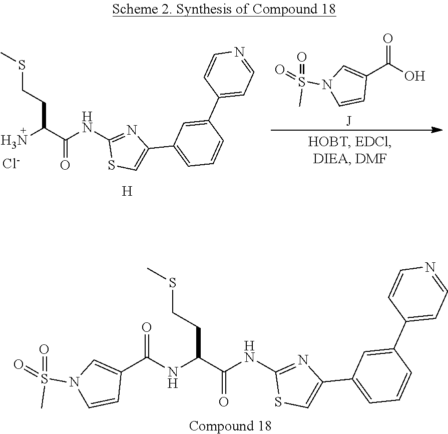

### Step 1: Preparation of (S)-1-(methylsulfonyl)-N-(4-(methylthio)-1-oxo-1-((4-(3-(pyridin-4-yl)phenyl)thiazol-2-yl)amino)butan-2-yl)-1H-pyrrole-3-carboxamide (compound 18)

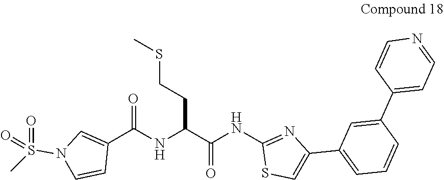 [SMILES: CSCC[C@H](NC(=O)c1ccn(S(C)(=O)=O)c1)C(=O)Nc1nc(-c2cccc(-c3ccncc3)c2)cs1]

To a mixture of (S)-4-(methylthio)-1-oxo-1-((4-(3-(pyridin-4-yl)phenyl)thiazol-2-yl)amino)butan-2-aminium chloride (2.00 g, 4.75 mmol) and 1-methylsulfonylpyrrole-3-carboxylic acid (0.899 g, 4.75 mmol) in DMF (20 mL) was added EDCI (1.37 g, 7.13 mmol), HOBt (0.963 g, 7.13 mmol), and N,N-diisopropylethylamine (3.31 mL, 19.00 mmol). After stirring for 3 h, the mixture was poured into water (100 mL) and the resulting precipitates were filtered. The solids were triturated in MeOH (20 mL) and the precipitate was collected by filtration. The solids were re-dissolved in DMSO (10 mL) and poured into MeOH (50 mL). The precipitates were filtered and lyophilized to give Compound 18 (2.05 g, 3.66 mmol, 77.01% yield) as white solids. LCMS (ESI) m/z [M+H]⁺=555.9. ¹H NMR (400 MHz, DMSO) δ 12.49 (s, 1H), 8.68-8.66 (m, 2H), 8.46 (d, J=7.2 Hz, 1H), 8.31-8.30 (m, 1H), 8.02-8.00 (m, 1H), 7.94-7.96 (m, 1H), 7.83 (s, 1H), 7.73-7.74 (m, 3H), 7.61-7.57 (m, 1H), 7.31-7.29 (m, 1H), 6.79-6.77 (m, 1H), 4.74-4.69 (m, 1H), 3.57 (s, 3H), 2.67-2.53 (m, 2H), 2.13-2.01 (m, 5H). SFC: AS-3-MeOH (DEA)-40-3 mL-35T.lcm, t=0.932 min, ee %=100%.

### Example 5. Effects of BRG1/BRM ATPase Inhibition on the Growth of Uveal Melanoma, Hematological Cancer, Prostate Cancer, Breast Cancer, and Ewing's Sarcoma Cell Lines

Procedure: All cell lines described above in Example 2 were also tested as described above with compound 18. In addition, the following cell lines were also tested as follows. Briefly, for Ewing's sarcoma cell lines (CADOES1, RDES, SKES1), retinoblastoma cell lines (WERIRB1), ALL cell lines (REH), AML cell lines (KASUMII), prostate cancer cell lines (P3, DU45, 22RV1), melanoma cell lines (SH4, SKMEL28, WM115, COLO829, SKMEL3, A375), breast cancer cell lines (MDAMB415, CAMA1, MCF7, BT474, HC1419, DU4475, BT549), B-ALL cell lines (SUPB15), CML cell lines (K562, MEG01), Burkitt's lymphoma cell lines (RAMOS2G64C10, DAUDI), mantle cell lymphoma cell lines (JEKO1, REC1), bladder cancer cell lines (HT1197), and lung cancer cell lines (SBC5), the above methods were performed with the following modifications: Cells were plated in 96 well plates, and the next day, BRG1/BRM ATPase inhibitor, compound 18, was dissolved in DMSO and added to the cells in a concentration gradient from 0 to 10 micromolar. At the time of cell splitting on days 3 and 7, cells were split into new 96 well plates, and fresh compound was added four hours after re-plating.

Table 2 lists the tested cell lines and growth media used.

TABLE 2
Cell Lines and Growth Media

| Cell Line | Source | Growth Media |
|---|---|---|
| 22RV1 | ATCC | RPMI1640 + 10% FBS |
| A375 | ATCC | DMEM + 10% FBS |
| BT474 | ATCC | Hybricare medium + |
| | | 1.5 g/L sodium |
| | | bicarbonate + 10% FBS |
| BT549 | ATCC | RPMI1640 + 0.023 IU/ml |
| | | insulin + 10% FBS |
| CADOES1 | DSMZ | RPMI1640 + 10% FBS |
| CAMA1 | ATCC | EMEM + 10% FBS |
| COLO829 | ATCC | RPMI1640 + 10% FBS |
| DAUDI | ATCC | RPMI1640 + 10% FBS |
| DU145 | ATCC | EMEM + 10% FBS |
| DU4475 | ATCC | RPMI1640 + 10% FBS |
| HCC1419 | ATCC | RPMI1640 + 10% FBS |
| HT1197 | ATCC | EMEM + 10% FBS |
| JEKO1 | ATCC | RPMI1640 + 20% FBS |
| K562 | ATCC | IMDM + 10% FBS |
| KASUMI1 | ATCC | RPMI1640 + 10% FBS |
| MCF7 | ATCC | EMEM + 0.01 mg/ml |
| | | bovine insulin + 10% FBS |
| MDAMB415 | ATCC | Leibovitz's L-15 + |
| | | 2 mM L-glutamine + |
| | | 10 mcg/ml insulin + |
| | | 10 mcg/ml glutathione + 15% FBS |
| MEG01 | ATCC | RPMI1640 + 10% FBS |
| PC3 | ATCC | F-12K + 10% FBS |
| RAMOS2G64C10 | ATCC | RPMI1640 + 10% FBS |
| RDES | ATCC | RPMI1640 + 15% FBS |
| REC1 | ATCC | RPMI1640 + 10% FBS |
| REH | ATCC | RPMI1640 + 10% FBS |
| SBC5 | JCRB | EMEM + 10% FBS |
| SH4 | ATCC | DMEM + 10% FBS |
| SKES1 | ATCC | McCoy's 5A + 15% FBS |
| SKMEL28 | ATCC | EMEM + 10% FBS |
| SKMEL3 | ATCC | McCoy's 5A + 15% FBS |
| SUPB15 | ATCC | IMDM + 4 mM L-glutamine + |
| | | 1.5 g/L sodium bicarbonate + |
| | | 0.05 mM 2-mercaptoethanol + 20% FBS |
| WERIRB1 | ATCC | RPMI1640 + 10% FBS |
| WM115 | ATCC | EMEM + 10% FBS |

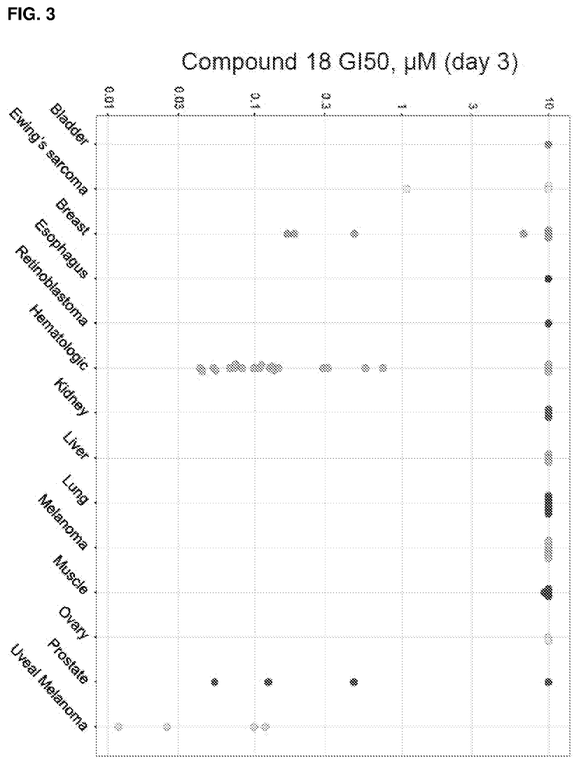

Results: As shown in FIG. 3, the uveal melanoma, hematologic cancer, prostate cancer, breast cancer, and Ewing's sarcoma cell lines were more sensitive to BRG1/BRM inhibition than the other tested cell lines. Inhibition of the uveal melanoma, hematologic cancer, prostate cancer, breast cancer, and Ewing's sarcoma cell lines was maintained through day 7.

### Example 6. Effects of BRG1/BRM ATPase Inhibition on the Growth of Cancer Cell Lines

Procedure: A pooled cell viability assay was performed using PRISM (Profiling Relative Inhibition Simultaneously in Mixtures) as previously described (“High-throughput identification of genotype-specific cancer vulnerabilities in mixtures of barcoded tumor cell lines”, Yu et al, Nature Biotechnology 34, 419-423, 2016), with the following modifications. Cell lines were obtained from the Cancer Cell Line Encyclopedia (CCLE) collection and adapted to RPMI-1640 medium without phenol red, supplemented with 10% heat-inactivated fetal bovine serum (FBS), in order to apply a unique infection and pooling protocol to such a big compendium of cell lines. A lentiviral spin-infection protocol was executed to introduce a 24 nucleotide-barcode in each cell line, with an estimated multiplicity of infection (MOI) of 1 for all cell lines, using blasticidin as selection marker. Over 750 PRISM cancer cell lines stably barcoded were then pooled together according to doubling time in pools of 25. For the screen execution, instead of plating a pool of 25 cell lines in each well as previously described (Yu et al.), all the adherent or all the suspension cell line pools were plated together using T25 flasks (100,000 cells/flask) or 6-well plates (50,000 cells/well), respectively. Cells were treated with either DMSO or compound in a 8-point 3-fold dose response in triplicate, starting from a top concentration of 10 μM. As control for assay robustness, cells were treated in parallel with two previously validated compounds, the pan-Raf inhibitor AZ-628, and the proteasome inhibitor bortezomib, using a top concentration of 2.5 μM and 0.039 μM, respectively.

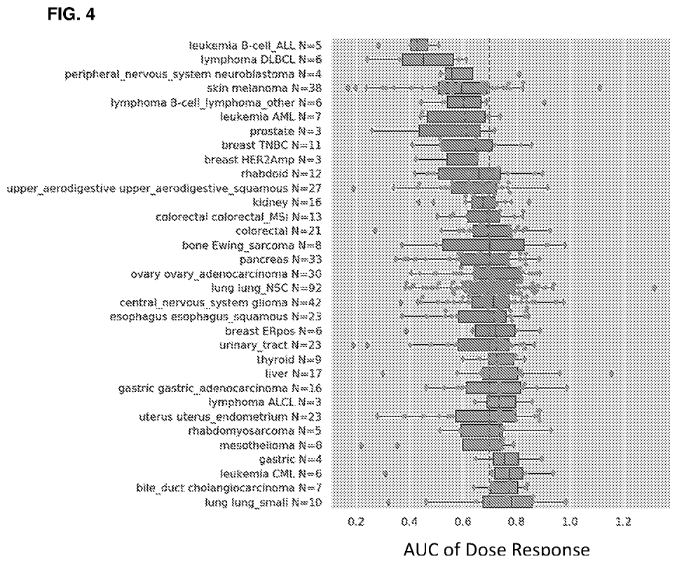

Following 3 days of treatment with compounds, cells were lysed, genomic DNA was extracted, barcodes were amplified by PCR and detected with Next-Generation Sequencing. Cell viability was determined by comparing the counts of cell-line specific barcodes in treated samples to those in the DMSO-control and Day 0 control. Dose-response curves were fit for each cell line and corresponding area under the curves (AUCs) were calculated and compared to the median AUC of all cell lines (FIG. 4). Cell lines with AUCs less than the median were considered most sensitive.

### Example 7. Effects of BRG1/BRM ATPase Inhibitors on the Growth of Uveal Melanoma Cell Lines

Procedure: Uveal melanoma cell lines (92-1, MP41, MP38, MP46) and non-small cell lung cancer cells (NCI-H1299) were plated into 96 well plates with growth media (see Table 1). BRG1/BRM ATPase inhibitor, compound 18, was dissolved in DMSO and added to the cells in a concentration gradient from 0 to 10 micromolar at the time of plating. Cells were incubated at 37° C. for 3 days. After three days of treatment, cell growth was measured with Cell-titer glow (Promega), and luminescence was read on an Envision plate reader (Perkin Elmer).

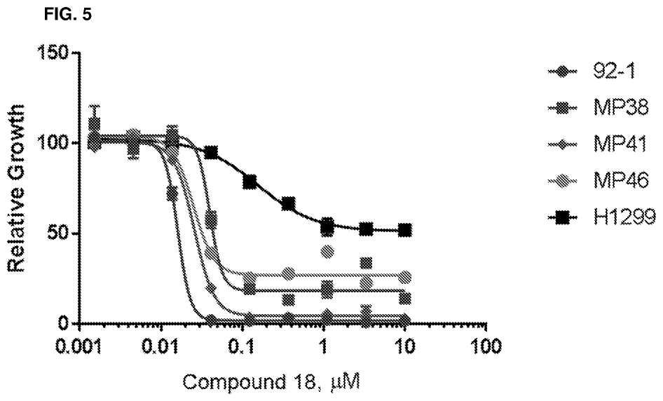

Results: As shown in FIG. 5, compound 18 resulted in potent growth inhibition in the uveal melanoma cell lines.

### Example 8. Comparison of BRG1/BRM Inhibitors to Clinical PKC and MEK Inhibitors in Uveal Melanoma Cell Lines

Procedure: Uveal melanoma cell lines, 92-1 or MP41, were plated in 96 well plates in the presence of growth media (see Table 1). BAF ATPase inhibitor (compound 18), PKC inhibitor (LXS196; MedChemExpress), and MEK inhibitor (Selumetinib; Selleck Chemicals) were dissolved in DMSO and added to the cells in a concentration gradient from 0 to 10 micromolar at the time of plating. Cells were incubated at 37° C. for 3 days. After three days of treatment, cell growth was measured with Cell-titer glow (Promega), and luminescence was read on an Envision plate reader (Perkin Elmer).

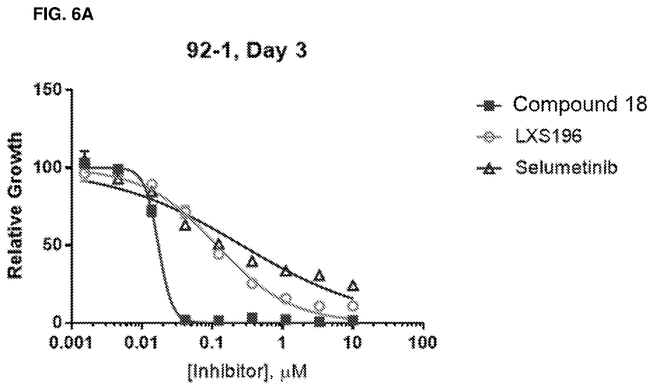

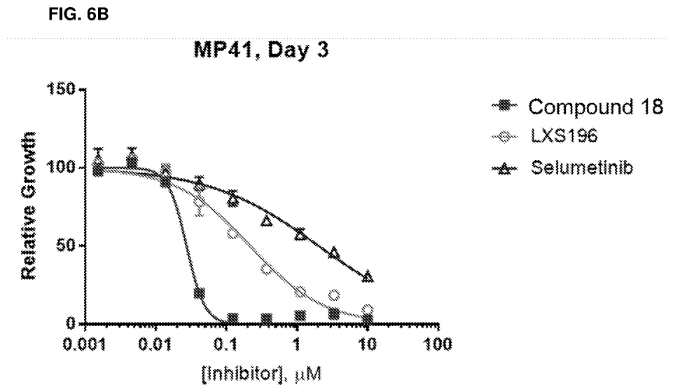

Results: As shown in FIG. 6A and FIG. 6B, compound 18 showed more potent effects on growth inhibition of uveal melanoma cells as compared to the clinical PKC and MEK inhibitors. Further, compound 18 was found to result in a faster onset of growth inhibition than the clinical PKC and MEK inhibitors.

### Example 9. BRG1/BRM ATPase Inhibitors are Effective at Inhibiting the Growth of PKC Inhibitor-Resistant Cells

Procedure: MP41 uveal melanoma cells were made resistant to the PKC inhibitor (LXS196; MedChemExpress), by long-term culture in growth media (see Table 1) containing increasing concentrations of the compound, up to 1 micromolar. After 3 months, sensitivity of the parental MP41 cells and the PKC inhibitor (PKCi)-resistant cells to the PKC inhibitor (LXS196) or the BRG1/BRM ATPase inhibitor (compound 18) was tested in a 7-day growth inhibition assay as described above in Example 2.

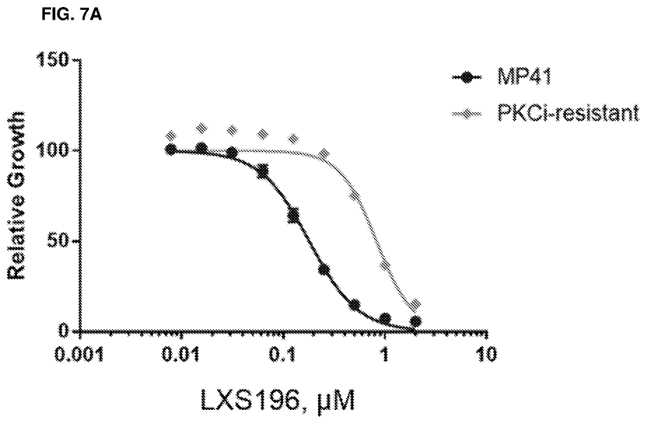

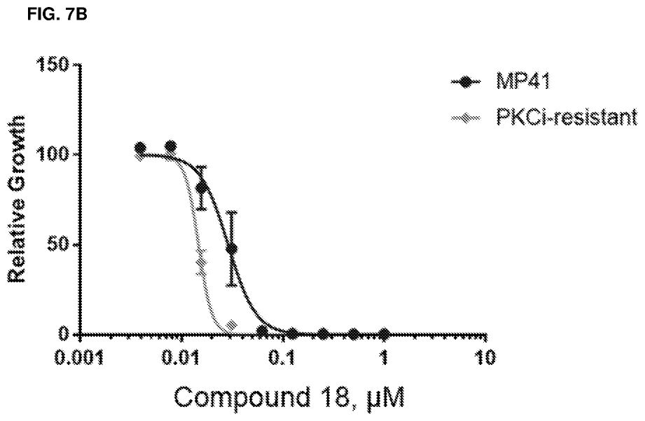

Results: While the PKCi-resistant cells could tolerate growth at higher concentrations of LXS196 than could the parental MP41 cell line (FIG. 7A), the BRG1/BRM ATPase inhibitor (compound 18) still resulted in strong growth inhibition of both the PKCi-resistant and parental cell lines (FIG. 7B). The PKCi-resistant cells were more sensitive to compound 18 than were the parental MP41 cells (FIG. 7B).

### Example 10. Synthesis of Compound 19

BRG1/BRM inhibitor compound 19 has the structure:

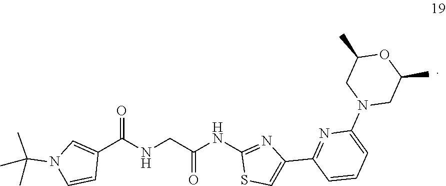 [SMILES: C[C@@H]1CN(c2cccc(-c3csc(NC(=O)CNC(=O)c4ccn(C(C)(C)C)c4)n3)n2)C[C@H](C)O1]

Compound 19 was synthesized as shown in Scheme 3 below.

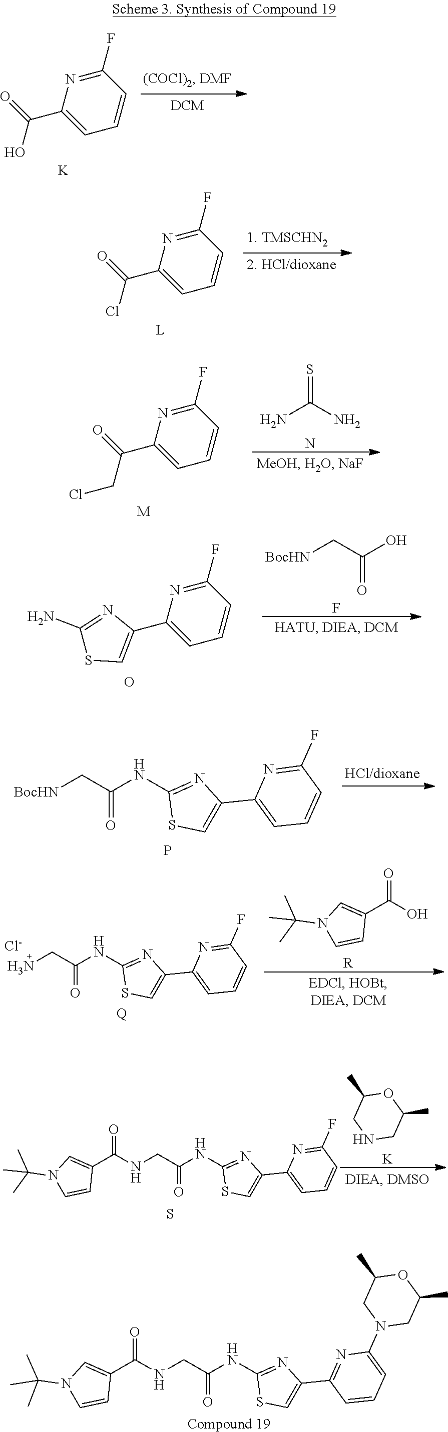

### Step 1: Preparation of 6-fluoropyridine-2-carbonyl chloride (Intermediate L)

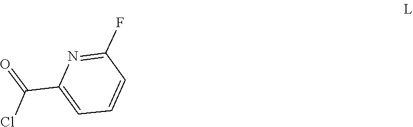

To a cooled (0° C.) solution of 6-fluoropyridine-2-carboxylic acid (50.00 g, 354.36 mmol) in dichloromethane (500 mL) and N,N-dimethylformamide (0.26 mL, 3.54 mmol) was added oxalyl chloride (155.10 mL, 1.77 mol). After complete addition of oxalyl chloride, the reaction mixture was warmed to room temperature and stirred for an additional 0.5 h. The mixture was subsequently concentrated in vacuo to give intermediate L (56.50 g) as white solids, which were used to next step without further purification.

Step 2: Preparation of 2-chloro-1-(6-fluoro-2-pyridyl)ethenone (Intermediate M)

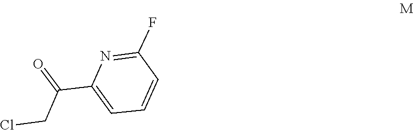 [SMILES: O=C(CCl)c1cccc(F)n1]

To a cooled (0° C.) mixture of intermediate L (56.00 g, 351.00 mmol) in 1,4-dioxane (800 mL) was added in a dropwise manner a solution of 2 M trimethylsilyl diazomethane in hexanes (351 mL). The resulting reaction mixture was stirred at 25° C. for 10 h. The reaction mixture was subsequently quenched with a solution of 4 M HCl in 1,4-dioxane (500 mL). After stirring for 2 h, the reaction solution was concentrated in vacuo to give an oil. The residue was diluted with saturated aqueous NaHCO₃ (500 mL) and extracted with EtOAc (3×200 mL). The combined organic layers were washed with brine (2×300 mL), dried over Na₂SO₄, filtered, and concentrated under reduced pressure to give intermediate M (35.50 g) as white solids, which was used to next step directly. LCMS (ESI) m/z: [M+H]⁺=173.8.

### Step 3: Preparation of 4-(6-fluoro-2-pyridyl)thiazol-2-amine (Intermediate O)

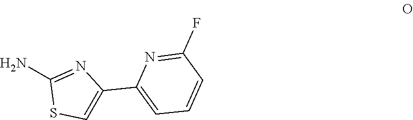 [SMILES: Nc1nc(-c2cccc(F)n2)cs1]

To a solution of intermediate M (35.50 g, 204.53 mmol) and thiourea (14.01 g, 184.07 mmol) in a mixture of MeOH (250 mL) and water (250 mL) at room temperature was added NaF (3.56 g, 84.82 mmol). After stirring for 30 min, the reaction mixture was partially concentrated in vacuo to remove MeOH. The resulting solution was acidified to pH ˜3 with 2 M aqueous HCl and extracted with EtOAc (3×200 mL). The combined organic layers were discarded, and the aqueous phase was basified with saturated aqueous NaHCO₃ (500 mL). After stirring for 30 min, the aqueous phase was extracted with EtOAc (3×325 mL). The combined organic layers were washed with brine (3×225 mL), dried over Na₂SO₄, filtered, and concentrated under reduced pressure. The solids were triturated with petroleum ether (300 mL), stirred at 25° C. for 10 min, and filtered. The solids were dried under vacuum to give intermediate O (28.00 g, 143.43 mmol, 70.13% yield, 100% purity) as white solids. LCMS (ESI) m/z: [M+H]⁺=195.8.; ¹H NMR (400 MHz, DMSO-d₆) δ 8.00-7.96 (m, 1H), 7.72 (d, J=7.2 Hz, 1H), 7.24 (s, 1H), 7.16 (s, 2H), 7.02 (d, J=8.0 Hz, 1H).

### Step 4: Preparation of tert-butyl N-[2-[[4-(6-fluoro-2-pyridyl)thiazol-2-yl]amino]-2-oxo-ethyl]carbamate (Intermediate P)

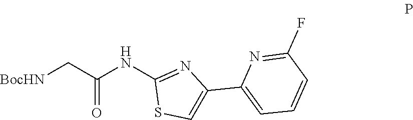 [SMILES: CC(C)(C)OC(=O)NCC(=O)Nc1nc(-c2cccc(F)n2)cs1]

To a solution of N-Boc-glycine (5.92 g, 33.81 mmol), HATU (12.86 g, 33.81 mmol), and N,N-diisopropylethylamine (21.41 mL, 122.94 mmol) in dichloromethane (100 mL) was added intermediate O (6.00 g, 30.74 mmol). After stirring for 2 h, the reaction mixture was concentrated. The resulting oil was diluted with water (100 mL) and subsequently extracted with EtOAc (4×60 mL). The combined organic layers were washed with brine (2×100 mL), dried over Na₂SO₄, filtered, and concentrated under reduced pressure to give solids. The solids were triturated with a 1:1 mixture of petroleum ether and MeOH (40 mL). After stirring at 25° C. for 20 minutes, the suspension was filtered, and the filter cake was washed with MTBE (20 mL). The solids were dried in vacuo to give intermediate P (7.70 g, 21.63 mmol, 70.4% yield, 99.0% purity) as white solids. LCMS (ESI) m/z: [M+H]⁺=353.1.

### Step 5: Preparation of 2-((4-(6-fluoropyridin-2-yl)thiazol-2-yl)amino)-2-oxoethan-1-aminium chloride (Intermediate Q)

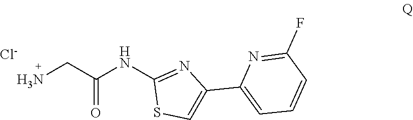 [SMILES: [Cl-] ; NCC(=O)Nc1nc(-c2cccc(F)n2)cs1]

A solution of intermediate P (5.40 g, 15.32 mmol) in 4 M HCl in 1,4-dioxane (35 mL) was stirred at 25° C. for 1.5 h. The reaction mixture was subsequently concentrated under vacuum to give intermediate Q (4.42 g) as white solids, which were used to next step directly without further purification. LCMS (ESI) m/z: [M+H]⁺=252.9.

### Step 6: Preparation of 1-tert-butyl-N-[2-[[4-(6-fluoro-2-pyridyl)thiazol-2-yl]amino]-2-oxo-ethyl]pyrrole-3-carboxamide (Intermediate S)

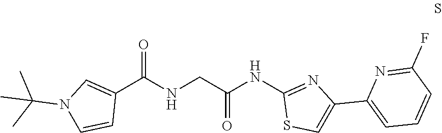 [SMILES: CC(C)(C)n1ccc(C(=O)NCC(=O)Nc2nc(-c3cccc(F)n3)cs2)c1]

To a solution of intermediate Q (3.00 g, 10.39 mmol), 1-tert-butylpyrrole-3-carboxylic acid (1.74 g, 10.39 mmol) and N,N-diisopropylethylamine (9.05 mL, 51.95 mmol) in dichloromethane (40 mL) was sequentially added HOBt (1.68 g, 12.47 mmol) and EDCI (2.39 g, 12.47 mmol). After stirring for 4 h, the mixture was concentrated in vacuo. The residue was diluted with water (250 mL) and extracted with EtOAc (3×200 mL). The combined organic layers were washed with brine (3×300 mL), dried over Na₂SO₄, filtered, and concentrated under reduced pressure. The resulting solids were triturated with a 1:1 mixture of MTBE/EtOAc (400 mL) and after stirring for 30 min, the suspension was filtered. The solids were washed with MTBE (3×85 mL) and dried under vacuum to give intermediate S (3.10 g, 7.64 mmol, 73.6% yield, 99.0% purity) as white solids. LCMS (ESI) m/z: [M+H]⁺=402.3.; ¹H NMR (400 MHz, DMSO-d6) δ 12.40 (s, 1H), 8.18-8.15 (m, 1H), 8.09-8.08 (m, 1H), 7.87-7.83 (m, 2H), 7.52 (s, 1H), 7.11 (d, J=8.0 Hz, 1H), 6.97 (m, 1H), 6.47 (s, 1H), 4.10 (d, J=5.6 Hz, 2H), 1.49 (s, 9H).

### Step 7: Preparation of 1-(tert-butyl)-N-(2-((4-(6-(cis-2,6-dimethylmorpholino)pyridin-2-yl)thiazol-2-yl)amino)-2-oxoethyl)-1H-pyrrole-3-carboxamide (compound 19)

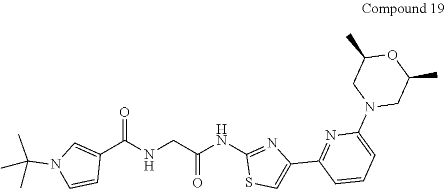 [SMILES: C[C@@H]1CN(c2cccc(-c3csc(NC(=O)CNC(=O)c4ccn(C(C)(C)C)c4)n3)n2)C[C@H](C)O1]

To a solution of intermediate S (0.100 g, 0.249 mmol) in DMSO (1 mL) was added N,N-diisopropylethylamine (0.130 mL, 0.747 mmol) and cis-2,6-dimethylmorpholine (0.057 g, 0.498 mmol). The resulting reaction mixture was stirred at 120° C. After 12 h, the solution was cooled to room temperature, diluted with MeOH (3 mL), and subsequently concentrated in vacuo. The resulting oil was purified by prep-HPLC (0.1% TFA; column: Luna C18 150*25 5 u; mobile phase: [water (0.075% TFA)-ACN]; B %: 30%-60%, 2 min). The appropriate fractions were collected and lyophilized to give Compound 19 (0.079 g, 0.129 mmol, 51.94% yield, 100% purity) as white solids. LCMS (ESI) m/z: [M+H]⁺=497.5.; ¹H NMR (400 MHz, DMSO-d₆) δ 12.27 (s, 1H), 8.17-8.14 (m, 1H), 7.75 (s, 1H), 7.63-7.59 (m, 1H), 7.51 (s, 1H), 7.25 (d, J=7.2 Hz, 1H), 6.96 (s, 1H), 6.79 (d, J=8.8 Hz, 1H), 6.47 (s, 1H), 4.24 (d, J=12.4 Hz, 2H), 4.08 (d, J=5.6 Hz, 2H), 3.64-3.61 (m, 2H), 2.44-2.38 (m, 2H), 1.49 (s, 9H), 1.18 (d, J=5.6 Hz, 6H).

### Example 11. BRG1/BRM ATPase Inhibitors Cause Uveal Melanoma Tumor Growth Inhibition In Vivo

Procedure: Nude mice (Envigo) were engrafted subcutaneously in the axillary region with 5×10⁶ 92-1 uveal melanoma cells in 50% Matrigel. Tumors were grown to a mean of ˜ 200 mm³, at which point mice were grouped and dosing was initiated. Mice were dosed once daily by oral gavage with vehicle (20% 2-Hydroxypropyl-β-Cyclodextrin) or increasing doses of compound 19. Tumor volumes and body weights were measured over the course of 3 weeks, and doses were adjusted by body weight to achieve the proper dose in terms of mg/kg. At this time, animals were sacrificed, and tumors were dissected and imaged.

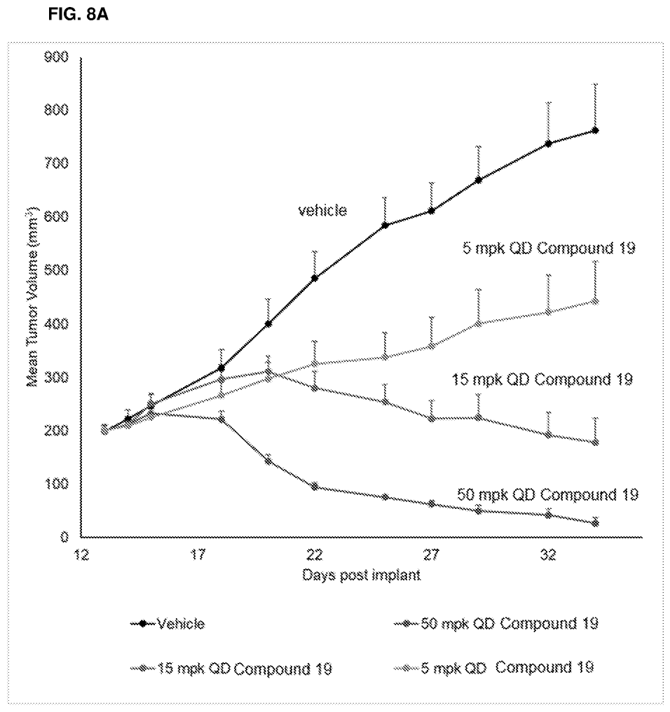

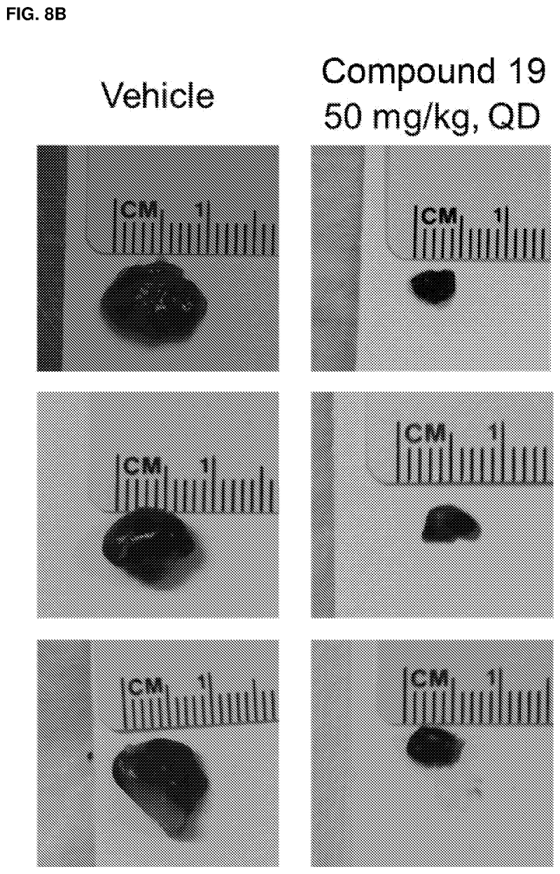

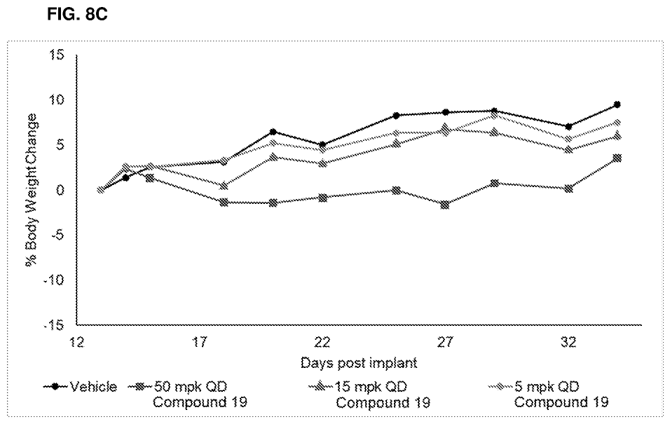

Results: Treatment with compound 19 led to tumor growth inhibition in a dose-dependent manner with tumor regression observed at the highest (50 mg/kg) dose. (FIG. 8A and FIG. 8B). All treatments were well tolerated based on % body weight change observed (FIG. 8C).

### Other Embodiments

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

What is claimed is:

1. A method of treating melanoma, prostate cancer, breast cancer, bone cancer, renal cell carcinoma, a hematologic cancer, or esophageal cancer in a subject in need thereof, the method comprising administering to the subject an effective amount of an agent that reduces the level and/or activity of BRG1 and/or BRM.

2. A method of reducing tumor growth of melanoma, prostate cancer, breast cancer, bone cancer, renal cell carcinoma, a hematologic cancer, or esophageal cancer in a subject in need thereof, the method comprising administering to the subject an effective amount of an agent that reduces the level and/or activity of BRG1 and/or BRM in the tumor.

3. A method of suppressing metastatic progression of melanoma, prostate cancer, breast cancer, bone cancer, renal cell carcinoma, a hematologic cancer, or esophageal cancer in a subject, the method comprising administering an effective amount of an agent that reduces the level and/or activity of BRG1 and/or BRM.

4. A method of suppressing metastatic colonization of melanoma, prostate cancer, breast cancer, bone cancer, renal cell carcinoma, a hematologic cancer, or esophageal cancer in a subject, the method comprising administering an effective amount of an agent that reduces the level and/or activity of BRG1 and/or BRM.

5. A method of reducing the level and/or activity of BRG1 and/or BRM in a melanoma, prostate cancer, breast cancer, bone cancer, renal cell carcinoma, hematologic cancer cell, or esophageal cancer cell, the method comprising contacting the cell with an effective amount of an agent that reduces the level and/or activity of BRG1 and/or BRM in the cell.

6. The method of claim 5, wherein the cell is in a subject.

7. The method of any one of claims 1 to 6, wherein the melanoma, prostate cancer, breast cancer, bone cancer, renal cell carcinoma, or hematologic cancer is metastatic.

8. The method of any one of claims 1 to 7, wherein the effective amount of the agent reduces the level and/or activity of BRG1 and/or BRM by at least 5% as compared to a reference.

9. The method of any one of claims 1 to 8, wherein the method further comprises administering to the subject or contacting the cell with an anticancer therapy.

10. The method of claim 9, wherein the anticancer therapy is a chemotherapeutic or cytotoxic agent, immunotherapy, surgery, radiotherapy, thermotherapy, or photocoagulation.

11. The method of claim 10, wherein the anticancer therapy is surgery.

12. The method of claim 10, wherein the anticancer therapy is a chemotherapeutic or cytotoxic agent.

13. The method of claim 12, wherein the chemotherapeutic or cytotoxic agent is an antimetabolite, antimitotic, antitumor antibiotic, asparagine-specific enzyme, bisphosphonates, antineoplastic, alkylating agent, DNA-Repair enzyme inhibitor, histone deacetylase inhibitor, corticosteroid, demethylating agent, immunomodulatory, janus-associated kinase inhibitor, phosphinositide 3-kinase inhibitor, proteasome inhibitor, or tyrosine kinase inhibitor.

14. The method of claim 12 or 13, wherein the one or more chemotherapeutic or cytotoxic agent is dacarbazine, temozolomide, cisplatin, treosulfan, fotemustine, IMCgp100, a CTLA-4 inhibitor, a PD-1 inhibitor, a PD-L1 inhibitor, a mitogen-activated protein kinase inhibitor, and/or a protein kinase C inhibitor.

15. The method of any one of claims 10 to 14, wherein the anticancer therapy and the agent that reduces the level and/or activity of BRG1 and/or BRM in a cell are administered within 28 days of each other and each in an amount that together are effective to treat the subject.

16. The method of any one of claims 1 to 15, wherein the subject or cancer has and/or has been identified as having a BRG1 loss of function mutation.

17. The method of any one of claims 1 to 15, wherein the subject or cancer has and/or has been identified as having a BRM loss of function mutation.

18. The method of any one of claims 1 to 17, wherein the melanoma, prostate cancer, breast cancer, bone cancer, renal cell carcinoma, hematologic cancer, or esophageal cancer has failed to respond to or progressed after administration of one or more chemotherapeutic or cytotoxic agents.

19. The method of any one of claims 1 to 18, wherein the melanoma, prostate cancer, breast cancer, bone cancer, renal cell carcinoma, hematologic cancer, or esophageal cancer is resistant to, or predicted to be resistant to one or more chemotherapeutic agents.

20. The method of claim 18 or 19, wherein the one or more chemotherapeutic or cytotoxic agents is dacarbazine, temozolomide, cisplatin, treosulfan, fotemustine, IMCgp100, a CTLA-4 inhibitor, a PD-1 inhibitor, a PD-L1 inhibitor, a mitogen-activated protein kinase inhibitor, and/or a protein kinase C inhibitor.

21. The method of any one of claims 1 to 20, wherein the melanoma, prostate cancer, breast cancer, bone cancer, renal cell carcinoma, hematologic cancer, or esophageal cancer is melanoma.

22. The method of claim 21, wherein the melanoma is uveal melanoma.

23. The method of claim 21, wherein the melanoma is mucosal melanoma.

24. The method of claim 21, wherein the melanoma is cutaneous melanoma.

25. The method of any one of claims 1 to 20, wherein the melanoma, prostate cancer, breast cancer, bone cancer, renal cell carcinoma, hematologic cancer, or esophageal cancer is a hematologic cancer.

26. The method of claim 25, wherein the hematologic cancer is multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia, diffuse large cell lymphoma, or non-Hodgkin's lymphoma.

27. The method of any one of claims 1 to 20, wherein the melanoma, prostate cancer, breast cancer, bone cancer, renal cell carcinoma, hematologic cancer, or esophageal cancer is prostate cancer.

28. The method of any one of claims 1 to 20, wherein the melanoma, prostate cancer, breast cancer, bone cancer, renal cell carcinoma, hematologic cancer, or esophageal cancer is breast cancer.

29. The method of claim 28, wherein the breast cancer is an ER positive breast cancer, an ER negative breast cancer, triple positive breast cancer, or triple negative breast cancer.

30. The method of any one of claims 1 to 20, wherein the melanoma, prostate cancer, breast cancer, bone cancer, renal cell carcinoma, hematologic cancer, or esophageal cancer is bone cancer.

31. The method of claim 30, wherein the bone cancer is Ewing's sarcoma.

32. The method of any one of claims 1 to 20, wherein the melanoma, prostate cancer, breast cancer, bone cancer, renal cell carcinoma, hematologic cancer, or esophageal cancer is renal cell carcinoma.

33. The method of claim 32, wherein the renal cell carcinoma is Microphthalmia Transcription Factor (MITF) family translocation renal cell carcinoma.

34. The method of any one of claims 1 to 20, wherein the melanoma, prostate cancer, breast cancer, bone cancer, renal cell carcinoma, hematologic cancer, or esophageal cancer is hematologic cancer.

35. The method of claim 34, wherein the hematologic cancer is multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia, diffuse large cell lymphoma, or non-Hodgkin's lymphoma.

36. The method of claim 35, wherein the acute lymphoblastic leukemia is T-cell acute lymphoblastic leukemia or B-cell acute lymphoblastic leukemia.

37. The method of any one of claims 1 to 20, wherein the melanoma, prostate cancer, breast cancer, bone cancer, renal cell carcinoma, hematologic cancer, or esophageal cancer is esophageal cancer.

38. The method of claim 37, wherein the esophageal cancer is esophageal adenocarcinoma or esophageal squamous-cell carcinoma.

39. The method of any one of claims 1 to 38, wherein the agent that reduces the level and/or activity of BRG1 and/or BRM in a cell is a small molecule compound, an antibody, an enzyme, and/or a polynucleotide.

40. The method of claim 39, wherein the agent that reduces the level and/or activity of BRG1 and/or BRM in a cell is an enzyme.

41. The method of claim 40, wherein the enzyme is a clustered regularly interspaced short palindromic repeats (CRISPR)-associated protein, a zinc finger nuclease (ZFN), a transcription activator-like effector nuclease (TALEN), or a meganuclease.

42. The method of claim 41, wherein the CRISPR-associated protein is CRISPR-associated protein 9 (Cas9) or CRISPR-associated protein 12a (Cas12a).

43. The method of claim 39, wherein the agent that reduces the level and/or activity of BRG1 and/or BRM in a cell is a polynucleotide.

44. The method of claim 43, wherein the polynucleotide is an antisense nucleic acid, a short interfering RNA, a short hairpin RNA, a micro RNA, a CRISPR/Cas 9 nucleotide, or a ribozyme.

45. The method of claim 39, wherein the agent that reduces the level and/or activity of BRG1 and/or BRM in a cell is a small molecule compound.

46. The method of claim 45, wherein the small molecule compound is a small molecule BRG1 and/or BRM inhibitor.

47. The method of claim 46, wherein the small molecule BRG1 and/or BRM inhibitor is a compound of Formula I.

48. The method of claim 46, wherein the small molecule BRG1 and/or BRM inhibitor is a compound of Formula III.

49. The method of claim 46, wherein the small molecule BRG1 and/or BRM inhibitor is a compound of Formula III.

50. The method of claim 46, wherein the small molecule BRG and/or BRM inhibitor has the structure of any one of compounds 1-16.

51. The method of claim 45, wherein the small molecule compound is a degrader of Formula IV.
